SEQUENCE LISTING (9 sequences)

SEQ ID NO: 1 - DNA, 1003 bp, Homo sapiens
  CDS (193)..(918)
gaattccggggagattccacactgtcagccccttctccaagatcagtacgtgggcctgactcctcctcggtgcccagctcagtattggcaactaggagagtagtgagattgaacttggccttgaggaacagctgcctctagagttggatcagacaagggtgctgagagccgggactcacaaccaaaggagaaatgagcaatccgcggtctttggaagaggagaaatatgatatgtcaggtgcccgcctggccctaatactgtgtgtcaccaaagcccgggaaggttccgaagaagacctggatgctctggaacacatgtttcggcagctgagattcgaaagcaccatgaaaagagaccccactgccgagcaattccaggaagagctggaaaaattccagcaggccatcgattcccgggaagatcccgtcagttgtgccttcgtggtactcatggctcacgggagggaaggcttcctcaagggagaagatggggagatggtcaagctggagaatctcttcgaggccctgaacaacaagaactgccaggccctgcgagctaagcccaaggtgtacatcatacaggcctgtcgaggagaacaaagggaccccggtgaaacagtaggtggagatgagattgtgatggtcatcaaagacagcccacaaaccatcccaacatacacagatgccttgcacgtttattccacggtagagggatacatcgcctaccgacatgatcagaaaggctcatgctttatccagaccctggtggatgtgttcacgaagaggaaaggacatatcttggaacttctgacagaggtgacccggcggatggcagaagcagagctggttcaagaaggaaaagcaaggaaaacgaaccctgaaatccaaagcaccctccggaaacggctgtatctgcagtagaagtagaaagaccaggaggagctttccttccagcattctttctgtctcacagaaatttagaagcagctcttacccggaattc

SEQ ID NO: 2 - protein, 242 aa, Homo sapiens
MSNPRSLEEEKYDMSGARLALILCVTKAREGSEEDLDALEHMFRQLRFESTMKRDPTAEQFQEELEKFQQAIDSREDPVSCAFVVLMAHGREGFLKGEDGEMVKLENLFEALNNKNCQALRAKPKVYIIQACRGEQRDPGETVGGDEIVMVIKDSPQTIPTYTDALHVYSTVEGYIAYRHDQKGSCFIQTLVDVFTKRKGHILELLTEVTRRMAEAELVQEGKARKTNPEIQSTLRKRLYLQ

SEQ ID NO: 3 - DNA, 27 bp, Homo sapiens
atgagcaatccgcggtctttggaagag

SEQ ID NO: 4 - protein, 9 aa, Homo sapiens
MSNPRSLEE

SEQ ID NO: 5 - protein, 9 aa, Homo sapiens
MDEFRENIT

SEQ ID NO: 6 - DNA, 24 bp, Homo sapiens
ggccctgcgagctaagcccaaggt

SEQ ID NO: 7 - DNA, 49 bp, Homo sapiens
aaaaagatctctactgcagatacagccgtttccggagggtgctttggat

SEQ ID NO: 8 - protein, 4 aa, Homo sapiens
DEVD

SEQ ID NO: 9 - protein, 242 aa, Homo sapiens
MDEFRENITEKYDMSGARLALILCVTKAREGSEEDLDALEHMFRQLRFESTMKRDPTAEQFQEELEKFQQAIDSREDPVSCAFVVLMAHGREGFLKGEDGEMVKLENLFEALNNKNCQALRAKPKVYIIQACRGEQRDPGETVGGDEIVMVIKDSPQTIPTYTDALHVYSTVEGYIAYRHDQKGSCFIQTLVDVFTKRKGHILELLTEVTRRMAEAELVQEGKARKTNPEIQSTLRKRLYLQ

(19) United States
(12) Patent Application Publication
(10) Pub. No.: US 2002/0081705 A1
(43) Pub. Date: Jun. 27, 2002

(54) Human caspase-14 compositions

(76) Inventor: John A. Mankovich, Andover, MA (US)

Correspondence Address: LAHIVE & COCKFIELD, 28 STATE STREET, BOSTON, MA, 02109, US

(21) Appl. No.: 09/845,028

(22) Filed: Apr. 27, 2001

Related U.S. Application Data
(60) Provisional application No. 60/199,962, filed on Apr. 27, 2000.

(51) Int. Cl. C12N 9/64; G01N 33/53; C12Q 1/37; C07H 21/04; C12P 21/02; C12N 5/06; C07K 16/40

(52) U.S. Cl. 435/226; 435/69.1; 435/325; 435/7.92; 530/388.26; 435/23; 536/23.2

(57) ABSTRACT

This invention provides the complete and correct nucleotide and amino acid sequences of human caspase-14. The invention provides an isolated human caspase-14 nucleic acid, wherein the nucleic acid comprises a coding region encoding human caspase-14 and the coding region comprises a nucleotide sequence ATG AGC AAT CCG COG TCT TTG GAA GAG (SEQ ID NO:3) at its 5′ end or the coding region encodes an amino acid sequence Met Ser Asn Pro Arg Ser Leu Glu Glu (SEQ ID NO:4) at its 5′ end. The invention also provides an isolated human caspase-14 protein comprising an amino acid sequence Met Ser Asn Pro Arg Ser Leu Glu Glu (SEQ ID NO:4) at its amino terminus. The invention also provides expression vectors, host cells and methods for making human caspase-14 proteins. The invention further provides fusion proteins, antibodies, non-human transgenic animals, and screening assays for identifying compounds which modulate human caspase-14.

### RELATED APPLICATIONS

This application claims the benefit of U.S. Provisional Application Ser. No. 60/199,962, filed Apr. 27, 2000. The entire contents of the above-referenced application are incorporated herein by this reference.

### BACKGROUND OF THE INVENTION

Caspases are a family of cysteine proteases that cleave following aspartate residues. The caspase family includes at least a dozen different members, which have been categorized into subfamilies (see e.g., Alnemri, E. S. et al. (1996) Cell 87:171; Salvesen, G. S. and Dixit, V. M. (1997) Cell 91:443-446; and Van de Craen, M. et al. (1997) FEBS Lett. 403:61-69). The caspase-1 subfamily includes caspase-1 (also known as IL-1 converting enzyme or ICE), caspase-4 (also known as ICErelII, TX and ICH2), caspase-5 (also known as ICErelIII and TY), caspase-1 (also known as Ich-3), caspase-12 and caspase-13 (also known as ERICE). The caspase-2 subfamily includes caspase-2 (also known as Ich-1). The caspase-3 subfamily includes caspase-3 (also known as Yama, CPP32 and apopain), caspase-6 (also known as Mch2), caspase-7 (also known as ICE-LAP3, Mch3 and CMH-1), caspase-8 (also known as FLICE, MACH and Mch5), caspase-9 (also known as ICE-LAP6 and Mch6) and caspase-10 (also known as FLICE2 and Mch4). Structurally, caspases typically comprise an amino-terminal prodomain, a large subunit (approximately 20 kD) and a small subunit (approximately 10 kD). Activation involves proteolytic processing between domains, followed by association of the large and small subunits to form a heterodimer (Thornberry, N. A. and Lazebnik, Y. (1998) Science 281:1312-1162).

Functionally, caspases are thought to be key mediators in the process of apoptotic cell death. Certain caspases also are involved in the proteolytic processing of precursor cytokines into mature biologically active forms, such as the processing of preIL-1β into mature IL-1β by ICE. Furthermore, certain caspases are capable of autocatalytic proteolysis to generate the mature form of the enzyme.

Another member of the caspase family, referred to as caspase-14, has been identified. The nucleotide and amino acid sequences of mouse caspase-14 have been described (see e.g., Van de Craen, M. et al. (1998) Cell Death Diff. 5:838-846; Hu, S. et al. (1998) Proc. Natl. Acad. Sci. USA 273:29648-29653; and Genbank Accession Numbers AF092997 and AJ007750). Additionally, a predicted amino acid sequence for human caspase-14 has been reported, based on use of a computer program to analyze a cosmid clone thought to contain the human caspase-14 gene (Van de Craen, M. et al. (1998) Cell Death Diff. 5:838-846). This predicted human caspase-14 protein was reported to have an amino terminal amino acid sequence of Met-Asp-Glu-Phe-Arg-Glu-Asn-Ile-Thr (SEQ ID NO:5).

### SUMMARY OF THE INVENTION

This invention provides the complete and correct nucleotide and amino acid sequences of human caspase-14. Contrary to what had previously been reported, the correct amino-terminal amino acid sequence of human caspase-14 is Met-Ser-Asn-Pro-Arg-Ser-Leu-Glu-Glu (SEQ ID NO:4), encoded by the nucleotide sequence ATG AGC AAT CCG CGG TCT TTG GAA GAG (SEQ ID NO:3). The full nucleotide sequence of a human caspase-14 cDNA is shown in SEQ ID NO:1, with the coding region for human caspase-14 protein corresponding to nucleotide positions 193-918. The full amino acid sequence of a human caspase-14 protein is shown in SEQ ID NO:2.

Accordingly, one aspect of the invention pertains to an isolated human caspase-14 nucleic acid, wherein the nucleic acid comprises a coding region encoding human caspase-14 and the coding region comprises a nucleotide sequence ATG AGC AAT CCG CGG TCT TTG GAA GAG (SEQ ID NO:3) at its 5′ end. In a preferred embodiment, the nucleic acid comprises the coding region of the nucleotide sequence of SEQ ID NO:1 (nucleotide positions 193-918). In another preferred embodiment, the nucleic acid comprises the nucleotide sequence of SEQ ID NO:1.

The invention also pertains to variants of human caspase-14. Accordingly, in one embodiment, the invention provides an isolated human caspase-14 nucleic acid, wherein the nucleic acid comprises a coding region encoding human caspase-14 and the coding region comprises a nucleotide sequence ATG AGC AAT CCG CGG TCT TTG GAA GAG (SEQ ID NO:3) at its 5′ end and wherein the nucleic acid has at least 95% nucleotide identity with the nucleotide sequence of SEQ ID NO:1. In another embodiment, the nucleic acid has at least 97% nucleotide identity with the nucleotide sequence of SEQ ID NO:1. In yet another embodiment, the nucleic acid has at least 99% nucleotide identity with the nucleotide sequence of SEQ ID NO:1.

In another embodiment, the invention provides an isolated human caspase-14 nucleic acid, wherein the nucleic acid comprises a coding region encoding human caspase-14 and the coding region encodes an amino acid sequence Met Ser Asn Pro Arg Ser Leu Glu Glu (SEQ ID NO:4) at its 5′ end. In a preferred embodiment, the nucleic acid encodes the amino acid sequence of SEQ ID NO:2.

In another embodiment, the nucleic acid comprises a coding region encoding human caspase-14, the coding region encodes an amino acid sequence Met Ser Asn Pro Arg Ser Leu Glu Glu (SEQ ID NO:4) at its 5′ end, and the nucleic acid encodes an amino acid sequence having at least 95% amino acid identity with the amino acid sequence of SEQ ID NO:2. More preferably, the nucleic acid encodes an amino acid sequence having at least 97% amino acid identity with the amino acid sequence of SEQ ID NO:2. Even more preferably, the nucleic acid encodes an amino acid sequence having at least 99% amino acid identity with the amino acid sequence of SEQ ID NO:2.

In other embodiments, the invention pertains to isolated nucleic acids comprising the complement of the above described nucleic acids. In another embodiment, the nucleic acid comprises a cDNA sequence. In still other embodiments, the invention pertains to isolated antisense nucleic acids comprising the nucleotide sequence of SEQ ID NO:3, and kits comprising a compound which selectively hybridizes to the nucleic acids of the invention.

The invention also pertains to expression vectors comprising the nucleic acids of the invention, and host cells comprising these expression vectors. Methods of producing human caspase-14 protein, using these vectors and hosts cells, are also encompassed. The method can involve, for example, culturing the host cell comprising the expression vector in a suitable culture medium until human caspase-14 protein is produced. The method can further involve isolating the human caspase-14 protein from the cells or the culture medium.

Another aspect of the invention pertains to human caspase-14 protein compositions. In one embodiment, the invention provides an isolated human caspase-14 protein comprising an amino acid sequence Met Ser Asn Pro Arg Ser Leu Glu Glu (SEQ ID NO:4) at its amino terminus. In a preferred embodiment, the protein comprises the amino acid sequence of SEQ ID NO:2. In another embodiment, the protein comprises an amino acid sequence Met Ser Asn Pro Arg Ser Leu Glu Glu (SEQ ID NO:4) at its amino terminus and further has at least 95% amino acid identity with the amino acid sequence of SEQ ID NO:2. In another embodiment, the protein comprises an amino acid sequence Met Ser Asn Pro Arg Ser Leu Glu Glu (SEQ ID NO:4) at its amino terminus and further has at least 97% amino acid identity with the amino acid sequence of SEQ ID NO:2. In yet another embodiment, the protein comprises an amino acid sequence Met Ser Asn Pro Arg Ser Leu Glu Glu (SEQ ID NO:4) at its amino terminus and further has at least 99% amino acid identity with the amino acid sequence of SEQ ID NO:2. In other embodiments, the invention pertains to kits comprising a compound which selectively binds to the human caspase-14 protein of the invention, and to pharmaceutical compositions comprising the human caspase-14 protein of the invention.

In other aspects, the invention pertains to fusion proteins comprising a human caspase-14 protein of the invention operatively linked to a non-caspase-14 protein or polypeptide; to antibodies (e.g., monoclonal human antibodies and antibodies linked to radioactive or cytotoxic agents) that bind to a human caspase-14 protein of the invention, wherein the antibody binds to the amino acid sequence Met Ser Asn Pro Arg Ser Leu Glu Glu (SEQ ID NO:4), and to non-human transgenic animals comprising the human caspase-14 nucleic acids of the invention.

In still other embodiment, the invention pertains to methods for identifying compounds which modulate human caspase-14 activity, bind the human caspase-14 protein, or modulate the interaction between the human caspase-14 protein and a target molecule. The invention further pertains to methods for identifying compounds which are capable of treating a disorder characterized by aberrant or abnormal human caspase-14 nucleic acid expression or human caspase-14 activity, and to methods for modulating apoptosis in a cell using human caspase-14 modulators.

### BRIEF DESCRIPTION OF THE DRAWINGS

FIG. 1 is a comparison of the sequence of SEQ ID NO:2 (referred to as “Caspase-14 NEW”) with that of the published human caspase-14 sequence (referred to as “Caspase-14 OLD”; SEQ ID NO:9), along with a consensus sequence.

FIG. 2 is a schematic diagram of the exon structure of the human caspase-14 gene, including the predicted published upstream exon.

### DETAILED DESCRIPTION OF THE INVENTION

This invention pertains to human caspase-14 compositions, such as isolated nucleic acid molecules encoding human caspase-14 and isolated human caspase-14 proteins, as well as methods of use therefore. The human compositions of the invention have the advantages that they comprise the correct amino-terminal sequence of naturally-occurring human caspase-14 and function optimally in human cells (compared with non-human caspase-14 compositions) and typically do not stimulate an immune response in humans.

So that the invention may be more readily understood, certain terms are first defined.

As used herein, the term “nucleic acid molecule” is intended to include DNA molecules (e.g., cDNA or genomic DNA) and RNA molecules (e.g., mRNA). The nucleic acid molecule may be single-stranded or double-stranded, but preferably is double-stranded DNA.

An used herein, an “isolated nucleic acid molecule” refers to a nucleic acid molecule that is free of gene sequences which naturally flank the nucleic acid in the genomic DNA of the organism from which the nucleic acid is derived (i.e., genetic sequences that are located adjacent to the gene for the isolated nucleic molecule in the genomic DNA of the organism from which the nucleic acid is derived). For example, in various embodiments, an isolated human caspase-14 nucleic acid molecule typically contains less than about 10 kb of nucleotide sequences which naturally flank the nucleic acid molecule in genomic DNA of the cell from which the nucleic acid is derived, and more preferably contains less than about 5 kb, 4 kb, 3 kb, 2 kb, 1 kb, 0.5 kb or 0.1 kb of naturally flanking nucleotide sequences. An “isolated” human caspase-14 nucleic acid molecule may, however, be linked to other nucleotide sequences that do not normally flank the human caspase-14 sequences in genomic DNA (e.g., the human caspase-14 nucleotide sequences may be linked to vector sequences). In certain preferred embodiments, an “isolated” nucleic acid molecule, such as a cDNA molecule, also may be free of other cellular material. However, it is not necessary for the human caspase-14 nucleic acid molecule to be free of other cellular material to be considered “isolated” (e.g., a human caspase-14 DNA molecule separated from other mammalian DNA and inserted into a bacterial cell would still be considered to be “isolated”).

As used herein, the term “hybridizes under high stringency conditions” is intended to describe conditions for hybridization and washing under which nucleotide sequences having substantial homology (e.g., typically greater than 70% homology) to each other remain stably hybridized to each other. A preferred, non-limiting example of high stringency conditions are hybridization in a hybridization buffer that contains 6×sodium chloride/sodium citrate (SSC) at a temperature of about 45° C. for several hours to overnight, followed by one or more washes in a washing buffer containing 0.2 ×SSC, 0.1% SDS at a temperature of about 50-65° C.

The term “%identity” as used in the context of nucleotide and amino acid sequences (e.g., when one amino acid sequence is said to be X% identical to another amino acid sequence) refers to the percentage of identical residues shared between the two sequences, when optimally aligned. To determine the percent identity of two nucleotide or amino acid sequences, the sequences are aligned for optimal comparison purposes (e.g., gaps may be introduced in one sequence for optimal alignment with the other sequence). The residues at corresponding positions are then compared and when a position in one sequence is occupied by the same residue as the corresponding position in the other sequence, then the molecules are identical at that position. The percent identity between two sequences, therefore, is a function of the number of identical positions shared by two sequences (i.e., % identity=# of identical positions/total # of positions×100).

The comparison of sequences and determination of percent identity between two sequences can be accomplished using a mathematical algorithm. In one embodiment, the percent identity between two amino acid sequences is determined using the Needleman and Wunsch (J. Mol. Biol. (48):444-453 (1970)) algorithm which has been incorporated into the GAP program in the GCG software package (available online through the Genetics Computer Group), using either a Blossom 62 matrix or a PAM250 matrix, and a gap weight of 16, 14, 12, 10, 8, 6, or 4 and a length weight of 1, 2, 3, 4, 5, or 6. In yet another embodiment, the percent identity between two nucleotide sequences is determined using the GAP program in the GCG software package (available online through the Genetics Computer Group), using a NWSgapdna.CMP matrix and a gap weight of 40, 50, 60, 70, or 80 and a length weight of 1, 2, 3, 4, 5, or 6. In another embodiment, the percent identity between two amino acid or nucleotide sequences is determined using the algorithm of Meyers, E. and Miller, W. (Comput. Appl. Biosci. 4:11-17 (1988)) which has been incorporated into the ALIGN program (version 2.0), using a PAM120 weight residue table, a gap length penalty of 12 and a gap penalty of 4.

The nucleic acid and protein sequences of the present invention can further be used as a “query sequence” to perform a search against public databases to, for example, identify other family members or related sequences. Such searches can be performed using the NBLAST and XBLAST programs (version 2.0) of Altschul et al. (1990) J. Mol. Biol. 215:403-10. BLAST nucleotide searches can be performed with the NBLAST program, score=100, wordlength=12 to obtain nucleotide sequences homologous to human caspase-14 nucleic acid molecules of the invention. BLAST protein searches can be performed with the XBLAST program, score=50, wordlength=3 to obtain amino acid sequences homologous to human caspase-14 protein molecules of the invention. To obtain gapped alignments for comparison purposes, Gapped BLAST can be utilized as described in Altschul et al. (1997) Nucleic Acids Res. 25(17):3389-3402. When utilizing BLAST and Gapped BLAST programs, the default parameters of the respective programs (e.g., XBLAST and NBLAST) can be used. See the website for the National Center for Biotechnology Information.

If multiple programs are used to compare sequences, the program that provides optimal alignment (i.e., the highest percent identity between the two sequences) is used for comparison purposes.

As used herein, a “naturally-occurring” nucleic acid molecule refers to an RNA or DNA molecule having a nucleotide sequence that occurs in nature (e.g., encodes a natural protein).

As used herein, an “antisense” nucleic acid comprises a nucleotide sequence which is complementary to a “sense” nucleic acid encoding a protein, e.g., complementary to the coding strand of a double-stranded cDNA molecule, complementary to an mRNA sequence or complementary to the coding strand of a gene. Accordingly, an antisense nucleic acid can hydrogen bond to a sense nucleic acid.

As used herein, the term “coding region” refers to regions of a nucleotide sequence comprising codons which are translated into amino acid residues, whereas the term “noncoding region” refers to regions of a nucleotide sequence that are not translated into amino acids (e.g., 5′ and 3′ untranslated regions).

As used herein, the term “vector” refers to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” or simply “expression vectors”. In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

As used herein, the term “host cell” is intended to refer to a cell into which a nucleic acid of the invention, such as a recombinant expression vector of the invention, has been introduced. The terms “host cell” and “recombinant host cell” are used interchangeably herein. It should be understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

As used herein, a “transgenic animal” refers to a non-human animal, preferably a mammal, more preferably a mouse, in which one or more of the cells of the animal includes a “transgene”. The term “transgene” refers to exogenous DNA which is integrated into the genome of a cell from which a transgenic animal develops and which remains in the genome of the mature animal, for example directing the expression of an encoded gene product in one or more cell types or tissues of the transgenic animal.

As used herein, a “homologous recombinant animal” refers to a type of transgenic non-human animal, preferably a mammal, more preferably a mouse, in which an endogenous gene has been altered by homologous recombination between the endogenous gene and an exogenous DNA molecule introduced into a cell of the animal, e.g., an embryonic cell of the animal, prior to development of the animal.

As used herein, an “isolated protein” refers to a protein that is separated from other proteins that occur in the organism from which the isolated protein is derived (i.e., other proteins that are present in, or made by, cells of the organism from which the isolated protein is derived). In certain preferred embodiments, an “isolated” protein also may be free of other materials, e.g., substantially free of other proteins, cellular material and culture medium when isolated from cells or produced by recombinant DNA techniques, or chemical precursors or other chemicals when chemically synthesized. However, it is not necessary for the human caspase-14 protein of the invention to be free of all other proteinaceous, cellular or chemical material to be considered “isolated” (e.g., a human caspase-14 protein separated from other human proteins and expressed by a bacterial cell in cell culture would still be considered to be “isolated”).

As used herein, the term “antibody” is intended to include immunoglobulin molecules and immunologically active portions of immunoglobulin molecules, i.e., molecules that contain an antigen binding site which specifically binds (immunoreacts with) an antigen, such as Fab and F(ab′)₂ fragments. The terms “monoclonal antibodies” and “monoclonal antibody composition”, as used herein, refer to a population of antibody molecules that contain only one species of an antigen binding site capable of immunoreacting with a particular epitope of an antigen, whereas the term “polyclonal antibodies” and “polyclonal antibody composition” refer to a population of antibody molecules that contain multiple species of antigen binding sites capable of interacting with a particular antigen. A monoclonal antibody compositions thus typically display a single binding affinity for a particular antigen with which it immunoreacts.

There is a known and definite correspondence between the amino acid sequence of a particular protein and the nucleotide sequences that can code for the protein, as defined by the genetic code (shown below). Likewise, there is a known and definite correspondence between the nucleotide sequence of a particular nucleic acid molecule and the amino acid sequence encoded by that nucleic acid molecule, as defined by the genetic code.

!GENETIC CODE

| | |
|---|---|
| Alanine (Ala, A) | GCA, GCC, GCG, GCT |
| Arginine (Arg, R) | AGA, ACG, CGA, CGC, CGG, |
| | CGT |
| Asparagine (Asn, N) | AAC, AAT |
| Aspartic acid (Asp, D) | GAC, GAT |
| Cysteine (Cys, C) | TGC, TGT |
| Glutamic acid (Glu, E) | GAA, GAG |
| Glutamine (Gln, Q) | CAA, CAG |
| Glycine (Gly, G) | GGA, GGC, GGG, GGT |
| Histidine (His, H) | CAC, CAT |
| Isoleucine (Ile, I) | ATA, ATC, ATT |
| Leucine (Leu, L) | CTA, CTC, CTG, CTT, TTA, |
| | TTG |
| Lysine (Lys, K) | AAA, AAG |
| Methionine (Met, M) | ATG |
| Phenylalanine (Phe, F) | TTC, TTT |
| Proline (Pro, P) | CCA, CCC, CCG, CCT |
| Serine (Ser, S) | AGC, AGT, TCA, TCC, TCG, |
| | TCT |
| Threonine (Thr, T) | ACA, ACC, ACG, ACT |
| Tryptophan (Trp, W) | TGG |
| Tyrosine (Tyr, Y) | TAC, TAT |
| Valine (Val, V) | GTA, GTC, GTG, GTT |
| Termination signal (end) | TAA, TAG, TGA |

An important and well known feature of the genetic code is its redundancy, whereby, for most of the amino acids used to make proteins, more than one coding nucleotide triplet may be employed (illustrated above). Therefore, a number of different nucleotide sequences may code for a given amino acid sequence. Such nucleotide sequences are considered functionally equivalent since they result in the production of the same amino acid sequence in all organisms (although certain organisms may translate some sequences more efficiently than they do others). Moreover, occasionally, a methylated variant of a purine or pyrimidine may be found in a given nucleotide sequence. Such methylations do not affect the coding relationship between the trinucleotide codon and the corresponding amino acid.

In view of the foregoing, the nucleotide sequence of a DNA or RNA molecule coding for a human caspase-14 protein of the invention (or any portion thereof) can be use to derive the human caspase-14 amino acid sequence, using the genetic code to translate the DNA or RNA molecule into an amino acid sequence. Likewise, for any human caspase-14 amino acid sequence, corresponding nucleotide sequences that can encode the human caspase-14 protein can be deduced from the genetic code (which, because of its redundancy, will produce multiple nucleic acid sequences for any given amino acid sequence). Thus, description and/or disclosure herein of a human caspase-14 nucleotide sequence should be considered to also include description and/or disclosure of the amino acid sequence encoded by the nucleotide sequence. Similarly, description and/or disclosure of a human caspase-14 amino acid sequence herein should be considered to also include description and/or disclosure of all possible nucleotide sequences that can encode the amino acid sequence.

Various aspects of the invention are described in further detail in the following subsections:

I. Isolated Nucleic Acid Molecules

One aspect of the invention pertains to isolated nucleic acid molecules that encode human caspase-14.

Contrary to what had previously been reported, the correct amino-terminal amino acid sequence of human caspase-14 is Met-Ser-Asn-Pro-Arg-Ser-Leu-Glu-Glu (SEQ ID NO:4), encoded by the nucleotide sequence ATG AGC AAT CCG CGG TCT TTG GAA GAG (SEQ ID NO:3). The full nucleotide sequence of a human caspase-14 cDNA is shown in SEQ ID NO:1, with the coding region for human caspase-14 protein corresponding to nucleotide positions 193-918. The full amino acid sequence of a human caspase-14 protein is shown in SEQ ID NO:2.

Accordingly, one aspect of the invention pertains to an isolated human caspase-14 nucleic acid, wherein the nucleic acid comprises a coding region encoding human caspase-14 and the coding region comprises a nucleotide sequence ATG AGC AAT CCG CGG TCT TTG GAA GAG (SEQ ID NO:3) at its 5′ end. In a preferred embodiment, the nucleic acid comprises the coding region of the nucleotide sequence of SEQ ID NO:1 (nucleotide positions 193-918). In another preferred embodiment, the nucleic acid comprises the nucleotide sequence of SEQ ID NO:1.

The invention further encompasses nucleic acid molecules that differ from SEQ ID NO:1 (and portions thereof) due to degeneracy of the genetic code and thus still encode the same human caspase-14 protein amino acid sequence. In one embodiment, the invention provides an isolated human caspase-14 nucleic acid, wherein the nucleic acid comprises a coding region encoding human caspase-14 and the coding region encodes an amino acid sequence Met Ser Asn Pro Arg Ser Leu Glu Glu (SEQ ID NO:4) at its 5′ end. In a preferred embodiment, the nucleic acid encodes the amino acid sequence of SEQ ID NO:2.

Additionally, it will be appreciated by those skilled in the art that DNA sequence polymorphisms that lead to changes in the amino acid sequences of human caspase-14 may exist within the human population. Such genetic polymorphism in the caspase-14 gene may exist among individuals within a population due to natural allelic variation. Such natural allelic variations can typically result in 1-5% variance in the nucleotide sequence of the a gene. Any and all such nucleotide variations and resulting amino acid polymorphisms in human caspase-14 that are the result of natural allelic variation and that do not alter the functional activity of human caspase-14 are intended to be within the scope of the invention. Accordingly, in one embodiment, the invention provides an isolated human caspase-14 nucleic acid, wherein the nucleic acid comprises a coding region encoding human caspase-14 and the coding region comprises a nucleotide sequence ATG AGC AAT CCG CGG TCT TTG GAA GAG (SEQ ID NO:3) at its 5′ end and wherein the nucleic acid has at least 95% nucleotide identity with the nucleotide sequence of SEQ ID NO:1. In another embodiment, the nucleic acid has at least 97% nucleotide identity with the nucleotide sequence of SEQ ID NO:1. In yet another embodiment, the nucleic acid has at least 99% nucleotide identity with the nucleotide sequence of SEQ ID NO:1. In other embodiments, the nucleic acid may have at least 96%, 98% or 99.5% nucleotide identity with the nucleotide sequence of SEQ ID NO:1.

In another embodiment, the nucleic acid comprises a coding region encoding human caspase-14, the coding region encodes an amino acid sequence Met Ser Asn Pro Arg Ser Leu Glu Glu (SEQ ID NO:4) at its 5′ end, and the nucleic acid encodes an amino acid sequence having at least 95% amino acid identity with the amino acid sequence of SEQ ID NO:2. More preferably, the nucleic acid encodes an amino acid sequence having at least 97% amino acid identity with the amino acid sequence of SEQ ID NO:2. Even more preferably, the nucleic acid encodes an amino acid sequence having at least 99% amino acid identity with the amino acid sequence of SEQ ID NO:2. In other embodiments, the nucleic acid may have at least 96%, 98% or 99.5% amino acid identity with the amino acid sequence of SEQ ID NO:2.

Additionally, in yet another embodiment, a nucleic acid molecule of the invention comprises a coding region encoding human caspase-14, the coding region comprises a nucleotide sequence ATG AGC AAT CCG CGG TCT TTG GAA GAG (SEQ ID NO:3) at its 5′ end, and the coding region hybridizes under high stringency hybridization conditions to a complement of the nucleic acid molecule of SEQ ID NO:1. In another embodiment, a nucleic acid molecule of the invention comprises a coding region encoding human caspase-14, the coding region encodes a polypeptide comprising an amino acid sequence Met Ser Asn Pro Arg Ser Leu Glu Glu (SEQ ID NO:4) at its 5′ end, and the coding region hybridizes under high stringency hybridization conditions to a complement of the nucleic acid molecule of SEQ ID NO:1.

A caspase-14-encoding nucleic acid of the invention can be isolated from a cDNA library using all or a part of SEQ ID NO:1 as a probe. More preferably, in view of the disclosure herein of the correct nucleotide sequence encoding human caspase-14 (SEQ ID NO:1), a nucleic acid of the invention can be isolated using standard molecular biology techniques, such as the polymerase chain reaction (PCR). For example, mRNA can be isolated from cells (e.g., by the guanidinium-thiocyanate extraction procedure of Chirgwin et al. (1979) Biochemistry 18:5294-5299) and cDNA can be prepared using reverse transcriptase (e.g., Moloney MLV reverse transcriptase, available from Gibco/BRL, Bethesda, Md.; or AMV reverse transcriptase, available from Seikagaku America, Inc., St. Petersburg, Fla.). Synthetic oligonucleotide primers can be designed based upon the nucleotide sequence shown in SEQ ID NO:1 for use in PCR to thereby amplify caspase-14 cDNA, or a portion thereof. A nucleic acid of the invention can be amplified from cDNA (or, alternatively, genomic DNA) using such oligonucleotide primers and standard PCR amplification techniques. The nucleic acid so amplified can be cloned into an appropriate vector and characterized by DNA sequence analysis. Alternatively, a probe comprising the nucleotide sequence of SEQ ID NO:1, or a portion thereof, can be used to screen a cDNA or genomic DNA library to thereby isolate caspase-14-encoding clones using standard library screening techniques. Furthermore, oligonucleotides of the caspase-14 sequence can be prepared by standard synthetic techniques, e.g., using an automated DNA synthesizer.

Another aspect of the invention pertains to antisense nucleic acids. Given the coding strand sequences encoding caspase-14 disclosed herein (SEQ ID NO:1), antisense nucleic acids of the invention can be designed according to the rules of Watson and Crick base pairing. The antisense nucleic acid molecule can be complementary to the entire coding region of caspase-14 mRNA, but more preferably is an oligonucleotide which is antisense to only a portion of the coding or noncoding region of caspase-14 mRNA. For example, the antisense oligonucleotide can be complementary to the region surrounding the translation start site of caspase 14 mRNA. An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a caspase-14 polypeptide to thereby inhibit expression of the polypeptide, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of an antisense nucleic acid molecule of the invention includes direct injection at a tissue site. Alternatively, an antisense nucleic acid molecule can be modified to target selected cells and then administered systemically. For example, for systemic administration, an antisense molecule can be modified such that it specifically binds to a receptor or an antigen expressed on a selected cell surface, e.g., by linking the antisense nucleic acid molecule to a peptide or an antibody which binds to a cell surface receptor or antigen. The antisense nucleic acid molecule can also be delivered to cells using the vectors described herein. To achieve sufficient intracellular concentrations of the antisense molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids. Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

II. Recombinant Expression Vectors and Host Cells

Another aspect of the invention pertains to vectors, preferably expression vectors, containing a nucleic acid encoding human caspase-14 of the invention (or a portion, subunit or homolog thereof). As used herein, the term “vector” refers to a nucleic acid molecule capable of transporting another nucleic acid to which it has been linked. One type of preferred vector is an episome, i.e., a nucleic acid capable of extra-chromosomal replication. Preferred vectors are those capable of autonomous replication and/or expression of nucleic acids to which they are linked. Vectors capable of directing the expression of genes to which they are operatively linked are referred to herein as “expression vectors”. In general, expression vectors of utility in recombinant DNA techniques are often in the form of “plasmids” which refer to circular double stranded DNA loops which, in their vector form are not bound to the chromosome. In the present specification, “plasmid” and “vector” are used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form “suitable for expression of the nucleic acid in a host cell”, which means that the recombinant expression vectors includes one or more regulatory sequences, selected on the basis of the host cells to be used for expression, which is operatively linked to the nucleic acid. “Operably linked” is intended to mean that the nucleotide sequence is linked to the regulatory sequence(s) in a manner which allows for expression of the nucleotide sequence (e.g., in an in vitro transcription/translation system or in a host cell when the vector is introduced into the host cell). The term “regulatory sequence” is intended to includes promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector may depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., human caspase-14 proteins, fusion proteins, subunits etc.).

The recombinant expression vectors of the invention can be designed for expression of human caspase-14 in prokaryotic or eukaryotic cells. For example, human caspase-14 can be expressed in bacterial cells such as E. coli, insect cells (using baculovirus expression vectors) yeast cells or mammalian cells. Suitable host cells are discussed further in Goeddel, Gene Expression Technology: Methods in Enzymology 185, Academic Press, San Diego, Calif. (1990). Alternatively, the recombinant expression vector may be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Amrad Corp., Melbourne, Australia), pMAL (New England Biolabs, Beverly, Mass.) and pRIT5 (Pharmacia, Piscataway, N.J.) which fuse glutathione S-transferase, maltose E binding protein, or protein A, respectively, to the target recombinant protein. For example, caspase-14 coding sequence can be cloned into an expression vector (e.g., an E. coli expression vector) that fuses a polyhistidine sequence (e.g., six histidine residues) to the N-terminus of caspase-14 coding sequence. The polyhistidine fusion moiety allows for purification of the caspase-14 protein on a nickel chelating column. Polyhistidine fusion expression vectors are commercially available (e.g., from Novagen).

Examples of suitable inducible non-fusion E. coli expression vectors include pTrc (Amann et al. (1988) Gene 69:301-315) and pET 11d (Studier et al. Gene Expression Technology: Methods in Enzymology 185, Academic Press, San Diego, Calif. (1990) 60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1). This viral polymerase is supplied by host strains BL21(DE3) or HMS174(DE3) from a resident λ prophage harboring a T7 gn1 gene under the transcriptional control of the lacUV 5 promoter.

One strategy to maximize recombinant protein expression in E. coli is to express the protein in a host bacteria with an impaired capacity to proteolytically cleave the recombinant protein (Gottesman, S., Gene Expression Technology: Methods in Enzymology 185, Academic Press, San Diego, Calif. (1990) 119-128). Another strategy is to alter the nucleic acid sequence of the nucleic acid to be inserted into an expression vector so that the individual codons for each amino acid are those preferentially utilized in E. coli (Wada et al. (1992) Nucleic Acids Res. 20:2111-2118). Such alteration of nucleic acid sequences of the invention can be carried out by standard DNA synthesis techniques.

In another embodiment, the caspase-14 expression vector is a yeast expression vector. Examples of vectors for expression in yeast S. cerivisae include pYepSec 1 (Baldari et al. (1987) EMBO J. 6:229-234), pMFa (Kurjan and Herskowitz (1982) Cell 30:933-943), pJRY88 (Schultz et al. (1987) Gene 54:113-123), and pYES2 (Invitrogen Corporation, San Diego, Calif.). In a preferred embodiment, caspase-14 is expressed in the methylotrophic yeast Hansenula polymorpha using an expression vector such as pMPT121, pFPMT121 or pRB (see e.g., Gellissen, G. et al. (1991) Biotechnology (NY) 9:291-295; and European Patent 0 173 378 BI). In these vectors, expression of a nucleic acid introduced into the vector is under the control of the MOX alcohol oxidase promoter (PMPT121) or the formate dehydrogenase promoter (pFPMT121 and pRB).

Alternatively, caspase-14 can be expressed in insect cells using baculovirus expression vectors. Baculovirus vectors available for expression of proteins in cultured insect cells (e.g., Sf 9 cells) include the pAc series (Smith et al. (1983) Mol. Cell Biol. 3:2156-2165) and the pVL series (Lucklow, V. A., and Summers, M. D. (1989) Virology 170:31-39).

In yet another embodiment, a nucleic acid of the invention is expressed in mammalian cells using a mammalian expression vector. Examples of mammalian expression vectors include pCDM8 (Seed, B. (1987) Nature 329:840) and pMT2PC (Kaufman et al. (1987), EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss (1990) Science 249:374-379) and the α-fetoprotein promoter (Campes and Tilghman (1989) Genes Dev. 3:537-546).

Moreover, inducible regulatory systems for use in mammalian cells are known in the art, for example systems in which gene expression is regulated by heavy metal ions (see e.g., Mayo et al. (1982) Cell 29:99-108; Brinster et al. (1982) Nature 296:39-42; Searle et al. (1985) Mol Cell. Biol. 5:1480-1489), heat shock (see e.g., Nouer et al. (1991) in Heat Shock Response, e.d. Nouer, L., CRC, Boca Raton, Fla., ppl 67-220), hormones (see e.g., Lee et al. (1981) Nature 294:228-232; Hynes et al. (1981) Proc. Natl. Acad. Sci. USA 78:2038-2042; Klock et al. (1987) Nature 329:734-736; Israel & Kaufman (1989) Nucleic Acids Res. 17:2589-2604; and PCT Publication No. WO 93/23431), FK506-related molecules (see e.g., PCT Publication No. WO 94/18317) or tetracyclines (Gossen, M. and Bujard, H. (1992) Proc. Natl. Acad. Sci. USA 89:5547-5551; Gossen, M. et al. (1995) Science 268:1766-1769; PCT Publication No. WO 94/29442; and PCT Publication No. WO 96/01313). Accordingly, in another embodiment, the invention provides a recombinant expression vector in which human caspase-14 DNA is operatively linked to an inducible eukaryotic promoter, thereby allowing for inducible expression of human caspase-14 protein in eukaryotic cells.

The invention further provides a recombinant expression vector comprising a DNA molecule of the invention cloned into the expression vector in an antisense orientation. That is, the DNA molecule is operatively linked to a regulatory sequence in a manner which allows for expression (by transcription of the DNA molecule) of an RNA molecule which is antisense to the coding region of the nucleotide sequence shown in SEQ ID NO:1. An “antisense” nucleic acid comprises a nucleotide sequence which is complementary to a “sense” nucleic acid, e.g., complementary to an mRNA sequence encoding a protein, constructed according to the rules of Watson and Crick base pairing. Accordingly, an antisense nucleic acid can hydrogen bond to a sense nucleic acid. For example, the antisense sequence complementary to a sequence of an mRNA can be complementary to a sequence found in the coding region of the mRNA or can be complementary to a 5′ or 3′ untranslated region of the mRNA. The binding of an antisense nucleic acid molecule to an mRNA molecule results in inhibition of translation of the mRNA molecule, thereby inhibiting production of the protein encoded by the mRNA molecule. Regulatory sequences operatively linked to a nucleic acid cloned in the antisense orientation can be chosen which direct the continuous expression of the antisense RNA molecule in a variety of cell types, for instance a viral promoter and/or enhancer, or regulatory sequences can be chosen which direct tissue or cell type specific expression of antisense RNA.

An antisense nucleic acid of the invention can be constructed using chemical synthesis and enzymatic ligation reactions using procedures known in the art. The antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest), as described above. The antisense expression vector, for example, can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see Weintraub, H. et al. Antisense RNA as a molecular tool for genetic analysis, Reviews—Trends in Genetics, Vol. 1(1) 1986.

In another embodiment, an antisense nucleic acid of the invention is a ribozyme. Ribozymes are catalytic RNA molecules with ribonuclease activity which are capable of cleaving a single-stranded nucleic acid, such as an mRNA, to which they have a complementary region. A ribozyme having specificity for a caspase-14 nucleic acid can be designed based upon the nucleotide sequence of a caspase-14 cDNA disclosed herein (i.e., SEQ ID NO:1). For example, a derivative of a Tetrahymena L-19 IVS RNA can be constructed in which the base sequence of the active site is complementary to the base sequence to be cleaved in a caspase-14-encoding mRNA. See for example Cech et al. U.S. Pat. No. 4,987,071; and Cech et al. U.S. Pat. No. 5,116,742. Alternatively, a caspase-14 nucleic acid of the invention could be used to select a catalytic RNA having a specific ribonuclease activity from a pool of RNA molecules. See for example Bartel, D. and Szostak, J. W. (1993) Science 261:1411-1418.

Another aspect of the invention pertains to recombinant host cells into which a recombinant expression vector of the invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell may be any prokaryotic or eukaryotic cell. For example, a caspase-14 protein may be expressed in bacterial cells such as E. coli , insect cells, yeast or mammalian cells (such as Chinese hamster ovary cells (CHO) or COS cells). Other suitable host cells are known to those skilled in the art.

Vector DNA is introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook et al. (Molecular Cloning: A Laboratory Manual, 2nd Edition, Cold Spring Harbor Laboratory press (1989)), and other laboratory textbooks.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin and methotrexate. Nucleic acid encoding a selectable marker may be introduced into a host cell on the same vector (e.g., plasmid) as that encoding caspase-14 or may be introduced on a separate vector (e.g., plasmid). Cells stably transfected with the introduced nucleic acid can be identified by drug selection (e.g., cells that have incorporated the selectable marker gene will survive, while the other cells die).

In one embodiment, a host cell of the invention is a fertilized oocyte or an embryonic stem cell into which caspase-14-coding sequences have been introduced. Such host cells can then be used to create non-human transgenic animals carrying caspase-14-coding nucleic acid in their genome. In one embodiment, a transgenic animal is created by introducing caspase-14 nucleic acid into the male pronuclei of a fertilized oocyte, e.g., by microinjection, and allowing the oocyte to develop in a pseudopregnant female foster animal. Intronic sequences and polyadenylation signals can also be included in the transgene to increase the efficiency of expression of the transgene. A tissue-specific regulatory sequence(s) can be operably linked to the caspase-14 transgene to direct expression of caspase-14 to particular cells. Methods for generating transgenic animals via embryo manipulation and microinjection, particularly animals such as mice, have become conventional in the art and are described, for example, in U.S. Pat. Nos. 4,736,866 and 4,870,009 and Hogan, B., Manipulating the Mouse Embryo (Cold Spring Harbor Laboratory Press, Cold Spring Harbor, N.Y., 1986). Similar methods are used for production of other transgenic animals. A transgenic founder animal can be identified based upon the presence of the caspase-14 transgene in its genome and/or expression of caspase-14 mRNA in tissues or cells of the animals. A transgenic founder animal can be used to breed additional animals carrying the transgene. Moreover, transgenic animals carrying a transgene encoding caspase-14 can further be bred to other transgenic animals carrying other transgenes.

In another embodiment, the transgenic animal has cells in which a gene corresponding to the non-human homolog of the caspase-14 gene has been functionally disrupted by homologous recombination. The term “homologous recombinant animal” as used herein is intended to describe an animal containing an endogenous gene which has been modified by homologous recombination between the endogenous gene and an exogenous DNA molecule introduced into a cell of the animal, e.g., an embryonic cell of the animal, prior to development of the animal. Preferably, the non-human homologous recombinant animal is a mouse.

To create such a homologous recombinant animal, a vector is prepared which contains at least a portion of a caspase-14 gene into which a deletion, addition or substitution has been introduced to thereby functionally disrupted the caspase-14 gene. The caspase-14 gene may be a human gene (e.g., from a human genomic clone isolated from a human genomic library screened with the nucleic acid of SEQ ID NO:1) or, more preferably, is a non-human homolog of a human caspase-14 gene. For example, a mouse caspase-14 gene can be isolated from a mouse genomic DNA library using the caspase-14 nucleic acid of SEQ ID NO:1 as a probe. In the homologous recombination vector, the functionally disrupted portion of the caspase-14 gene is flanked at its 5′ and 3′ ends by additional nucleic acid of the caspase-14 gene to allow for homologous recombination to occur between the exogenous caspase-14 gene carried by the vector and an endogenous caspase-14 gene in an embryonic stem cell. The additional flanking caspase-14 nucleic acid is of sufficient length for successful homologous recombination with the endogenous gene. Typically, at least one kilobase and more preferably several kilobases of flanking DNA (both at the 5′ and 3′ ends) are included in the vector (see e.g., Thomas, K. R. and Capecchi, M. R. (1987) Cell 51:503 for a description of homologous recombination vectors). The vector is introduced into an embryonic stem cell line (e.g., by electroporation) and cells in which the introduced caspase-14 gene has homologously recombined with the endogenous caspase-14 gene are selected (see e.g., Li, E. et al. (1992) Cell 69:915). The selected cells are then injected into a blastocyst of an animal (e.g., a mouse) to form aggregation chimeras (see e.g., Bradley, A. in Teratocarcinomas and Embryonic Stem Cells: A Practical Approach, Robertson, E. J., ed. (IRL, Oxford, 1987) pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene.

III. Isolated Caspase-14 Proteins

Another aspect of the invention pertains to isolated human caspase-14 proteins. In a preferred embodiment, the invention provides an isolated human caspase-14 protein comprising an amino acid sequence Met Ser Asn Pro Arg Ser Leu Glu Glu (SEQ ID NO:4) at its amino terminus. In a preferred embodiment, the protein comprises the amino acid sequence of SEQ ID NO:2. In another embodiment, the protein comprises an amino acid sequence Met Ser Asn Pro Arg Ser Leu Glu Glu (SEQ ID NO:4) at its amino terminus and further has at least 95% amino acid identity with the amino acid sequence of SEQ ID NO:2. In another embodiment, the protein comprises an amino acid sequence Met Ser Asn Pro Arg Ser Leu Glu Glu (SEQ ID NO:4) at its amino terminus and further has at least 97% amino acid identity with the amino acid sequence of SEQ ID NO:2. In yet another embodiment, the protein comprises an amino acid sequence Met Ser Asn Pro Arg Ser Leu Glu Glu (SEQ ID NO:4) at its amino terminus and further has at least 99% amino acid identity with the amino acid sequence of SEQ ID NO:2. In yet other embodiments, the protein comprises an amino acid sequence Met Ser Asn Pro Arg Ser Leu Glu Glu (SEQ ID NO:4) at its amino terminus and further has at least 96%, 98% or 99.5% amino acid identity with the amino acid sequence of SEQ ID NO:2.

Additionally, the invention provides proteolytic fragments of human caspase-14, such as caspase-14 p20 and p10 subunits. Examination of known cleavage sites in caspase family members allows for the identification of a predicted cleavage site between Aspartate-154 and Serine-155 of caspase-14. Accordingly, the invention further provides novel proteolytic fragments of human caspase-14 generated by cleavage of the full-length protein between Asp-154 and Ser-155. In one embodiment, a proteolytic fragment of the invention comprises an amino acid sequence Met Ser Asn Pro Arg Ser Leu Glu Glu (SEQ ID NO:4) at its amino terminus and further has at least 95% amino acid identity with amino acid positions 1-154 of SEQ ID NO:2, more preferably 96%, 97%, 98%, 99%, 99.5% or 100% amino acid identity with amino acid positions 1-154 of SEQ ID NO:2. This proteolytic fragment can be encoded by a nucleic acid molecule comprising a nucleotide sequence ATG AGC AAT CCG CGG TCT TTG GAA GAG (SEQ ID NO:3) at its 5′ end and further comprising a nucleotide sequence having at least 95%, 96%, 97%, 98%, 99%, 99.5% or 100% nucleotide identity with the nucleotide positions of SEQ ID NO:1 encoding amino acids 1-154 of SEQ ID NO:2. Such nucleic acid molecules are also encompassed by the invention. Other aspects of the invention include a proteolytic fragment of human caspase-14 comprising amino acids 155-242 of SEQ ID NO:2, and a nucleic acid molecule encoding amino acids 155-242 of SEQ ID NO:2.

The caspase-14 proteins, or subunits thereof, are preferably produced by recombinant DNA techniques. For example, a nucleic acid molecule encoding the protein is cloned into an expression vector (as described above), the expression vector is introduced into a host cell (as described above) and the caspase-14 protein is expressed in the host cell. The caspase-14 protein can then be isolated from the cells by an appropriate purification scheme using standard protein purification techniques. Alternative to recombinant expression, a caspase-14 polypeptide can be synthesized chemically using standard peptide synthesis techniques. Alternatively, a native caspase-14 protein can be isolated from cells (e.g., human cells), for example using an anti-caspase-14 antibody (discussed further below).

The invention still further provides caspase-14 fusion proteins. As used herein, a caspase-14 “fusion protein” comprises a caspase-14 polypeptide fused to a heterologous (i.e., non-caspase-14) polypeptide. The heterologous polypeptide may be fused to the N-terminus or C-terminus of the caspase-14 protein (or subunit thereof). Purification of a caspase-14 protein can be facilitated by the expression of the caspase-14 protein as a fusion protein, wherein the heterologous polypeptide of the fusion protein facilitates purification of the fusion protein. For example, as described in above in Section II, a nucleic acid encoding a caspase-14 protein (or portion or subunit thereof) can be cloned into a prokaryotic expression vector encoding a fusion moiety (i.e., heterologous polypeptide), such that the resultant expression vector encodes a fusion protein comprising the caspase-14 protein and the fusion moiety. Examples of suitable fusion moieties that facilitate protein purification include glutathione S-transferase, maltose E binding protein, protein A and polyhistidine. The polyhistidine sequence of the fusion protein facilitates purification of the fusion protein by affinity chromatography using a Ni²⁺ metal resin. The fusion protein may additionally contain a cleavage site, e.g., for Factor Xa, thrombin or enterokinase, between the fusion moiety (e.g., polyhistidine sequence) and the caspase-14 sequence to allow for removal of the fusion moiety after purification of the fusion protein, if desired. In a preferred embodiment, the caspase-14 fusion protein comprises six histidine residues fused to the N-terminus of a caspase-14.

Preferably, a fusion protein is produced by recombinant expression of a fusion gene encoding the fusion protein. Techniques for making fusion genes are known to those skilled in the art. Essentially, the joining of various DNA fragments coding for different polypeptide sequences is performed in accordance with conventional techniques, for example employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons: 1992). Moreover, many expression vectors are commercially available that already encode a fusion moiety (e.g., polyhistidine sequence, GST sequence, etc.). A caspase-14-encoding nucleic acid can be cloned into such an expression vector such that the fusion moiety is linked in-frame to the caspase-14 protein.

An isolated human caspase-14 protein, or subunit or fragment thereof, can be used as an immunogen to generate antibodies that bind a human caspase-14 protein using standard techniques for polyclonal and monoclonal antibody preparation. Accordingly, anti-human caspase-14 antibodies are also encompassed by the invention. The term “antibody” as used herein refers to immunoglobulin molecules and immunologically active portions of immunoglobulin molecules, i.e., molecules that contain an antigen binding site which specifically binds (immunoreacts with) an antigen, such as human caspase-14. The invention provides polyclonal and, more preferably, monoclonal antibodies that bind human caspase-14. The term “monoclonal antibody” or “monoclonal antibody composition”, as used herein, refers to a population of antibody molecules that contain only one species of an antigen binding site capable of immunoreacting with a particular epitope of human caspase-14. A monoclonal antibody composition thus typically displays a single binding affinity for a particular protein with which it immunoreacts.

Additionally, recombinant anti-human caspase-14 antibodies, such as chimeric and humanized monoclonal antibodies, comprising both human and non-human portions, which can be made using standard recombinant DNA techniques, are within the scope of the invention.

An antibody of the invention is typically prepared by immunizing a suitable subject with an appropriate immunogenic preparation of a human caspase-14 protein and isolating an antibody that binds the protein. An appropriate immunogenic preparation can contain, for examples, recombinantly expressed human caspase-14 protein or a chemically synthesized human caspase-14 peptide. The preparation can further include an adjuvant, such as Freund's complete or incomplete adjuvant, or similar immunostimulatory agent. Immunization of a suitable subject (e.g., rabbit, goat, mouse or other mammal, etc.) with an immunogenic human caspase-14 preparation induces a polyclonal anti-human caspase-14 antibody response. The anti-human caspase-14 antibody titer in the immunized subject can be monitored over time by standard techniques, such as with an enzyme linked immunosorbent assay (ELISA) using immobilized human caspase-14. If desired, the antibody molecules directed against human caspase-14 can be isolated from the mammal (e.g., from the blood) and further purified by well known techniques, such as protein A chromatography to obtain the IgG fraction. At an appropriate time after immunization, e.g., when the anti-human caspase-14 antibody titers are highest, antibody-producing cells can be obtained from the subject and used to prepare monoclonal antibodies.

A monoclonal anti-human caspase-14 antibody can be prepared and isolated using a technique which provides for the production of antibody molecules by continuous cell lines in culture. These include, but are not limited to, the hybridoma technique originally described by Kohler and Milstein (1975) Nature 256:495-497) (see also, Brown et al. (1981) J. Immunol. 127:539-46; Brown et al. (1980) J. Biol. Chem. 255:4980-83; Yeh et al. (1976) Proc. Natl. Acad. Sci. USA 76:2927-31; and Yeh et al. (1982) Int. J. Cancer 29:269-75), and the more recent human B cell hybridoma technique (Kozbor et al. (1983) Immunol. Today 4:72), EBV-hybridoma technique (Cole et al. (1985) Monoclonal Antibodies and Cancer Therapy, Alan R. Liss, Inc., pp. 77-96), and trioma techniques. The technology for producing monoclonal antibody hybridomas is well known (see generally Kenneth, R. H. in Monoclonal Antibodies: A New Dimension In Biological Analyses, Plenum Publishing Corp., New York, N.Y. (1980); Lerner, E. A. (1981) Yale J. Biol. Med. 54:387-402; Gefter, M. L. et al. (1977) Somatic Cell Genet. 3:231-36). Briefly, an immortal cell line (typically myeloma cells) is fused to lymphocytes (typically splenocytes) from a mammal immunized with an immunogenic preparation of the present invention, as described above, and the culture supernatants of the resulting hybridoma cells are screened to identify a hybridoma producing a monoclonal antibody that binds human caspase-14.

Any of the many well known protocols used for fusing lymphocytes and immortalized cell lines can be applied for the purpose of generating an anti-human caspase-14 monoclonal antibody (see, e.g., Galfre, G. et al. (1977) Nature 266:55052; Gefter et al. (1977) supra; Lerner (1981) supra; Kenneth, Monoclonal Antibodies, supra). Moreover, the ordinary skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines may be used as a fusion partner according to standard techniques, e.g., the P3-NS1/1-Ag4-1, P3-x63-Ag8.653 or Sp2/O-Ag14 myeloma lines. These myeloma lines are available from the American Type Culture Collection (ATCC), Rockville, Md. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind human caspase-14, e.g., using a standard ELISA assay.

Alternative to preparing monoclonal antibody-secreting hybridomas, a monoclonal anti-human caspase-14 antibody can be identified and isolated by screening a recombinant combinatorial immunoglobulin library (e.g., an antibody phage display library) with human caspase-14 to thereby isolate immunoglobulin library members that bind human caspase-14. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, McCafferty et al. International Publication No. WO 92/01047, U.S. Pat. No. 5,969,108 and EP 589,877 (describing in particular display of scFv), Ladner et al. U.S. Pat. No. 5,223,409, No. 5,403,484, No. 5,571,698, No. 5,837,500 and EP 436,597 (describing, for example, pIII fusion); Dower et al. International Publication No. WO 91/17271, U.S. Pat. No. 5,427,908, U.S. Pat. No. 5,580,717 and EP 527,839 (describing in particular display of Fab); Winter et al. International Publication WO 92/20791 and EP 368,684 (describing in particular cloning of immunoglobulin variable domain sequences); Griffiths et al. U.S. Pat. No. 5,885,793 and EP 589,877 (describing in particular isolation of human antibodies to human antigens using recombinant libraries); Garrard et al. International Publication No. WO 92/09690 (describing in particular phage expression techniques); Knappik et al. International Publication No. WO 97/08320 (describing the human recombinant antibody library HuCal); and Salfeld et al. International Publication No. WO 97/29131 (describing the preparation of a recombinant human antibody to a human antigen, as well as in vitro affinity maturation of the recombinant antibody).

Other descriptions of recombinant antibody library screenings can be found in scientific publications such as Fuchs et al. (1991) Biotechnology (NY) 9:1369-1372; Hay et al. (1992) Hum. Antibod. Hybridomas 3:81-85; Huse et al. (1989) Science 246:1275-1281; Griffiths et al. (1993) EMBO J. 12:725-734; Hawkins et al. (1992) J. Mol. Biol. 226:889-896; Clackson et al. (1991) Nature 352:624-628; Gram et al. (1992) Proc. Natl. Acad. Sci. USA 89:3576-3580; Garrard et al. (1991) Biotechnology (NY) 9:1373-1377; Hoogenboom et al. (1991) Nucleic Acids Res. 19:4133-4137; Barbas et al. (1991) Proc. Natl. Acad. Sci. USA 88:7978-7982; McCafferty et al. (1990) Nature 348:552-554; and Knappik et al. (2000) J. Mol. Biol. 296:57-86.

Chimeric and humanized versions of an anti-human caspase-14 monoclonal antibody are also within the scope of the invention. Such chimeric and humanized monoclonal antibodies can be produced by recombinant DNA techniques known in the art, for example using methods described in Robinson et al. International Patent Publication PCT/US86/02269; Akira et al. European Patent Application 184,187; Taniguchi, M., European Patent Application 171,496; Morrison et al. European Patent Application 173,494; Neuberger et al. PCT Application WO 86/01533; Cabilly et al. U.S. Pat. No. 4,816,567; Cabilly et al. European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Cancer Res. 47:999-1005; Wood et al. (1985) Nature 314:446-449; and Shaw et al. (1988) J. Natl. Cancer Inst. 80:1553-1559); Morrison, S. L. (1985) Science 229:1202-1207; Oi et al. (1986) Biotechniques 4:214; Winter U.S. Pat. No. 5,225,539; Jones et al. (1986) Nature 321:552-525; Verhoeyen et al. (1988) Science 239:1534; and Beidler et al. (1988) J. Immunol. 141:4053-4060.

An anti-human caspase-14 antibody (e.g., monoclonal antibody) can be used to isolate a human caspase-14 protein by standard techniques, such as affinity chromatography or immunoprecipitation. An anti-human caspase-14 antibody can facilitate the purification of natural human caspase-14 from cells and of recombinantly produced human caspase-14 expressed in host cells. Moreover, an anti-human caspase-14 antibody can be used to detect human caspase-14 protein (e.g., in a cellular lysate or cell supernatant) in order to evaluate the abundance and pattern of expression of the human caspase-14 protein or a fragment of a human caspase-14 protein. Anti-human caspase-14 protein antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S, or ³H.

Further, an antibody (or fragment thereof) may be conjugated to a therapeutic agent such as a cytotoxin, or a radioactive material. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

The conjugates of the invention can be used for modifying a given biological response; however, the therapeutic agent is not to be construed as limited to classical chemical therapeutic agents. For example, the therapeutic agent may be a protein or polypeptide possessing a desired biological activity. Such proteins may include, for example, a toxin such as abrin, ricin A, pseudomonas exotoxin, or diphtheria toxin; a protein such as tumor necrosis factor, alpha-interferon, beta-interferon, nerve growth factor, platelet derived growth factor, tissue plasminogen activator; or, biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other growth factors.

The therapeutic agent may also be a radioactive material (e.g., a radionuclide). Exemplary radionuclides include ⁹⁰Y, ¹⁸⁸Re, ²¹At, ²¹²Bi and the like. Other reactor-produced radionuclides are useful in the practice of these embodiments of the present invention, if they are able to bind in amounts delivering a therapeutically effective amount of radiation to the target. A therapeutically effective amount of radiation ranges from about 1500 to about 10,000 cGy, depending upon several factors known to those of skill in the art.

Techniques for conjugating such therapeutic moiety to antibodies are well known, see, e.g., Arnon et al. “Monoclonal Antibodies for Immunotargeting of Drugs in Cancer Therapy”, in Monoclonal Antibodies and Cancer Therapy, Reisfeld et al. (eds.), pp. 243-56 (Alan R. Liss, Inc. 1985); Hellstrom et al. “Antibodies For Drug Delivery”, in Controlled Drug Delivery (2^{nd} Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological and Clinical Applications, Pinchera et al. (eds.), pp. 475-506 (1985); “Analysis, Results, and Future Prospective of the Therapeutic Use of Radiolabeled Antibody in Cancer Therapy”, in Monoclonal Antibodies for Cancer Detection and Therapy, Baldwin et al. (eds.), pp. 303-16 (Academic Press 1985), and Thorpe et al. “The Preparation and Cytotoxic Properties of Antibody-Toxin Conjugates”, Immunol. Rev. 62:119-58 (1982). Alternatively, an antibody can be conjugated to a second antibody to form an antibody heteroconjugate as described by Segal in U.S. Pat. No. 4,676,980.

IV. Pharmaceutical Compositions

The human caspase-14 nucleic acid molecules, proteins (including fragments of human caspase-14) and anti-human caspase-14 antibodies (also referred to herein as “active compounds”) of the invention can be incorporated into pharmaceutical compositions suitable for administration to a subject, e.g., a human. Such compositions typically comprise the nucleic acid molecule, protein, or antibody and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, such media can be used in the compositions of the invention. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor ELTM (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for exarnple, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a human caspase-14 protein or anti-human caspase-14 antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

V. Uses and Methods of the Invention

The human caspase-14 protein of the invention is a cysteinyl aspartate-specific proteinase that is a member of the caspase family of proteases. The human caspase-14 protein of the invention displays structural features that evidence its membership in the caspase family. The pentapeptide motif QACRG (positions 130-134 of SEQ ID NO:2) is conserved in all known caspases except for substitutions at position 133 in caspase-8, -9 and -10. This motif contains the catalytic nucleophile, Cys132. A second catalytic residue, His89, is believed to function as a general acid-base during catalysis, and His89 as well as the adjacent Gly90 are strictly conserved among caspases. Gln130 and Ser180 form part of the S1 site and contact substrate P1 side chains. Each are strictly conserved among caspases with the exception of the conservative substitution of threonine for Ser180 in caspase-8. The substrate-binding residue Arg179 is also strictly conserved. This residue forms parts of both the S1 and S3 subsites of caspases, contacting substrate P1 and P3 side chains and directing the absolute specificity of all caspases for Asp in P1 of substrates as well as the preference for Glu in P3 that is common to many caspases. Arg179 of caspase-1 is highly conserved among caspases, and makes a second contact with the P3 side chains of substrates, and may be represented by Arg29 of caspase-14. The caspase-14 sequence VIKDS (positions 151-155 of SEQ ID NO:2), while not strictly conserved among caspases, aligns well with known proteolytic maturation sites of caspases between the p20 and p10 subunits (for cleavage between Asp154 and Ser155). This particular sequence is typical of caspase cleavage sequences in many known substrates and matches the known substrate specificities of many caspases, and so is identified as a predicted site of proteolytic maturation by caspase-14 and/or other caspases. Cys270, Leu272, Pro277, Lys278 and Asp326 are all strictly conserved among all caspases including caspase-14. They are distant from the active site, in the interface between the p20 and p10 subunits of activated caspases, and may have important structural roles. Leu353 is also strictly conserved, located in the hydrophobic core of caspases, and may also have an important structural role. Thus, for caspase-14 proteins of the invention that may differ in amino acid sequence from that disclosed in SEQ ID NO:2 (e.g., caspase-14 proteins having 95% or greater amino acid identity to SEQ ID NO:2), it is important to maintain the conserved residues discussed above.

The human caspase-14 of the invention can be used as a protease to cleave substrates. For example, the recombinantly expressed murine homologue of the human caspase-14 of the invention has been shown to be capable of cleaving the fluorometric caspase substrate Ac-DEVD-Afc in an in vitro protease assay (described in Hu, S. et al. (1998) J. Biol. Chem. 273:29648-29653). The human caspase-14 protein of the invention can be similarly expressed recombinantly and used to cleave caspase substrates.

Moreover, the human caspase-14 of the invention, when overexpressed in cells, can be used to induce apoptosis in the cells. For example, the murine homologue of the human caspase-14 of the invention has been shown to be capable of inducing apoptosis in MCF7 cells when an expression vector encoding the protease is transfected into the cells and overexpressed therein (described in Hu, S. et al. (1998) J. Biol. Chem. 273:29648-29653). Nucleic acid encoding the human caspase-14 protein of the invention can be similarly transfected into cells and overexpressed therein to induce apoptosis in the cells. Accordingly, another aspect of the invention pertains to a method for modulating apoptosis in a cell comprising contacting the cell with an agent that modulates activity of human caspase-14 in the cell. In one embodiment, the agent stimulates human caspase-14 activity. This agent may be, for example, a human caspase-14-encoding nucleic acid. Nucleic acid encoding human caspase-14 can be introduced into cells (e.g., by transfection of a human caspase-14 cDNA) to stimulate apoptosis in the cells. Thus, a nucleic acid molecule encoding human caspase-14 (e.g., cDNA) can be transfected into target cells as a “suicide” gene in situations where it is desirable to stimulate death of the target cells. Human caspase-14 may be used to stimulate apoptosis in cells for research purposes (e.g., cell ablation studies) and for therapeutic purposes. For example, a human caspase-14 nucleic acid can be introduced into diseased cells, such as cancer cells to reduce tumor growth, smooth muscle cells to inhibit restenosis, fibroblasts to inhibit fibrosis and rheumatoid arthritis, synovial cells to inhibit rheumatoid arthritis and T and/or B lymphocytes to inhibit autoimmune diseases such as rheumatoid arthritis, multiple sclerosis and systemic lupus erythematosus. A recombinant expression vector of the invention can be used to express human caspase-14 in cells. Tissue-specific and/or regulated expression of human caspase-14 can be accomplished through the use of appropriate tissue-specific and/or inducible transcriptional regulatory elements within the expression vector.

Moreover, alternative to introducing a human caspase-14-encoding nucleic acid molecule into cells as a means to stimulate apoptosis in the cells, the cells can be treated with an agent that stimulates endogenous human caspase-14 activity in the cells (referred to herein as a “human caspase-14 activator”). A human caspase-14 activator may stimulate endogenous human caspase-14 activity, for example, by increasing the transcription of the human caspase-14 gene, the translation of the human caspase-14 mRNA, or the enzymatic activity of the human caspase-14 protein. Such human caspase-14 activators can be identified using screening assays provided by the invention, described in greater detail below.

In another embodiment of the method of modulating apoptosis, the cell can be contacted with an agent that inhibits human caspase-14 activity to thereby inhibit apoptosis in the cells. Accordingly, inhibitors of human caspase-14 activity may be useful in the treatment of disease conditions involving cell death by acting to inhibit or slow down this process. Examples of such disease conditions that may be amenable to treatment with an inhibitor of caspase-14 activity include neural and muscular degenerative diseases, myocardial infarction, stroke, virally-induced cell death, aging, inflammation, autoimmune diseases and AIDS. An inhibitor of human caspase-14 may act on the enzymatic activity of the human caspase-14 protein or may inhibit the production of the human caspase-14 protein (e.g., transcription of the human caspase-14 gene or translation of the human caspase-14 mRNA). For example, one type of human caspase-14 inhibitor provided by the invention is an antisense nucleic acid that binds to human caspase-14 mRNA to thereby inhibit the production of human caspase-14 protein in cells. Such an antisense nucleic acid can be introduced into target cells (e.g., transfected into cells) to inhibit human caspase-14 activity in the cells. Alternatively, agents that inhibit human caspase-14 activity can be identified using screening assays provided by the invention, described in greater detail below.

In view of the foregoing, yet another aspect of the invention pertains to methods for identifying agents that modulate (e.g., inhibit or stimulate) human caspase-14 protease activity. Accordingly, the invention provides a method for identifying a modulator of human caspase-14 protease activity comprising:

a) contacting a human caspase-14 protein of the invention with a potential substrate for the protein in the presence of a test agent under proteolytic conditions;

b) measuring human caspase-14 protease activity against the substrate in the presence of the test agent; and

c) identifying a modulator of human caspase-14 protease activity.

In one embodiment of the method, an inhibitor of human caspase-14 protease activity is identified. For example, human caspase-14 protein is contacted with a potential substrate for the human caspase-14 protein in the presence of a test agent under proteolytic conditions (i.e., in the absence of the test agent, the human caspase-14 exhibits proteolytic activity against the known human caspase-14 substrate under these conditions). The proteolytic activity of the human caspase-14 protein against the substrate in the presence of the test agent is then determined. A decrease in the amount of human caspase-14 proteolytic activity in the presence of the test agent relative to the amount of human caspase-14 proteolytic activity in the absence of the test agent indicates that the test agent is a human caspase-14 protease inhibitor.

In another embodiment of the method, an activator of human caspase-14 protease activity is identified. This method is similar to that described above for identifying human caspase-14 inhibitors (i.e., a human caspase-14 protein is incubated with a substrate in the presence of a test agent and the proteolytic activity of the human caspase-14 protein against the substrate is determined). However, in this embodiment, an increase in the amount of human caspase-14 proteolytic activity in the presence of the test agent relative to the amount of human caspase-14 proteolytic activity in the absence of the test agent indicates that the test agent is a human caspase-14 protease activator.

Human caspase-14 proteins for use in the screening assays of the invention can be prepared as described above in Sections II and III. For example, in one embodiment, the protein is derived from a recombinantly expressed human caspase-14 comprising the amino acid sequence of SEQ ID NO:2. In one embodiment, the human caspase-14 protein is derived from a polyhistidine fusion protein expressed in E. coli . Methods for expressing caspase-14 in E. coli as a polyhistidine fusion protein are described in detail in Hu, S. et al. (1998) J. Biol. Chem. 273:29648-29653.

Suitable human caspase-14 substrates for use in the screening assays include peptide substrates and derivatives thereof. A preferred peptide substrate is derived from the tetrapeptide Asp-Glu-Val-Asp (DEVD) (SEQ ID NO:8), modified preferably with an acetyl group at the amino-terminal end and with a detectable substance at the carboxy-terminal end, such as p-nitroanilide (a chromogenic substrate), amino-4-methylcoumarin (a fluorogenic substrate) and AFc (a fluorogenic substrate). Cleavage of such peptide substrates can be detected spectrophotometrically. Additionally, whole proteins containing a caspase-14 cleavage site can be used as substrates for human caspase-14. Whole proteins can be labeled (e.g., with ³⁵S-methionine) and their cleavage products can be directly detected (e.g., by SDS-PAGE and autoradiography). Alternatively, cleavage of whole proteins can be detected indirectly (e.g., using an antibody that binds a specific cleavage product).

Moreover, the nucleic acid molecules, proteins, modulators, and antibodies described herein can be used in drug screening assays and/or diagnostic assays. The isolated nucleic acid molecules of the invention can be used to express human caspase-14 protein (e.g., via a recombinant expression vector in a host cell or in gene therapy applications), to detect human caspase-14 mRNA (e.g., in a biological sample) or a naturally occurring or recombinantly generated genetic mutation in a human caspase-14 gene, and to modulate human caspase-14 activity, as described further below. In addition, the human caspase-14 proteins can be used to screen drugs or compounds which modulate caspase-14 protein activity as well as to treat disorders characterized by insufficient production of caspase-14 or production of caspase-14 forms which have decreased activity compared to wild type caspase-14. Moreover, the anti-caspase-14 antibodies of the invention can be used to detect and isolate a human caspase-14 polypeptide, particularly caspase-14 present in a biological sample, and to modulate caspase-14 activity.

The invention provides methods for identifying compounds or agents which can be used to treat disorders characterized by (or associated with) aberrant or abnormal human caspase-14 nucleic acid expression and/or human caspase-14 protein activity. These methods are also referred to herein as drug screening assays and typically include the step of screening a candidate/test compound or agent to be an agonist or antagonist of human caspase-14, and specifically for the ability to interact with (e.g., bind to) a human caspase-14 protein, to modulate the interaction of a human caspase-14 protein and a target molecule (e.g., substrate), and/or to modulate human caspase-14 nucleic acid expression and/or human caspase-14 protein activity. Candidate/test compounds or agents which have one or more of these abilities can be used as drugs to treat disorders characterized by aberrant or abnormal human caspase-14 nucleic acid expression and/or human caspase-14 protein activity. Candidate/test compounds include, for example, 1) peptides such as soluble peptides, including Ig-tailed fusion peptides and members of random peptide libraries (see, e.g., Lam, K. S. et al. (1991) Nature 354:82-84; Houghten, R. et al. (1991) Nature 354:84-86) and combinatorial chemistry-derived molecular libraries made of D- and/or L-configuration amino acids; 2) phosphopeptides (e.g., members of random and partially degenerate, directed phosphopeptide libraries, see, e.g., Songyang, Z. et al. (1993) Cell 72:767-778); 3) antibodies (e.g., polyclonal, monoclonal, humanized, anti-idiotypic, chimeric, and single chain antibodies as well as Fab, F(ab′)₂, Fab expression library fragments, and epitope-binding fragments of antibodies); and 4) small organic and inorganic molecules (e.g., molecules obtained from combinatorial and natural product libraries).

In one embodiment, the invention provides assays for screening candidate/test compounds which interact with (e.g., bind to) human caspase-14 protein. Typically, the assays are recombinant cell based or cell-free assays which include the steps of combining a human caspase-14 protein or a bioactive fragment thereof, and a candidate/test compound, e.g., under conditions which allow for interaction of (e.g., binding of) the candidate/test compound to the human caspase-14 or fragment thereof to form a complex, and detecting the formation of a complex, in which the ability of the candidate compound to interact with (e.g., bind to) the human caspase-14 protein or fragment thereof is indicated by the presence of the candidate compound in the complex. Formation of complexes between the human caspase-14 protein and the candidate compound can be quantitated, for example, using standard immunoassays.

In another embodiment, the invention provides screening assays to identify candidate/test compounds which modulate (e.g., stimulate or inhibit) the interaction (and most likely human caspase-14 activity as well) between a human caspase-14 protein and a molecule (target molecule) with which the human caspase-14 normally interacts. Examples of such target molecules include substrates of caspase-14 and polypeptides in the same signaling path as human caspase-14, e.g., polypeptides which may function upstream (including both stimulators and inhibitors of activity) or downstream of the human caspase-14 protein in, for example, an apoptotic signaling pathway or in a pathway involving proteolytic processing. Typically, the assays are recombinant cell based or cell-free assays which include the steps of combining a cell expressing a human caspase-14 protein, or a bioactive fragment thereof, a human caspase-14 target molecule (e.g., a human caspase-14 substrate) and a candidate/test compound, e.g., under conditions wherein but for the presence of the candidate compound, the human caspase-14 protein or biologically active portion thereof interacts with (e.g., binds to) the target molecule, and detecting the formation of a complex which includes the human caspase-14 protein and the target molecule or detecting the interaction/reaction of the human caspase-14 protein and the target molecule. Detection of complex formation can include direct quantitation of the complex by, for example, measuring inductive effects of the human caspase-14 protein. A statistically significant change, such as a decrease, in the interaction of the human caspase-14 protein and target molecule (e.g., in the formation of a complex between the human caspase-14 protein and the target molecule) in the presence of a candidate compound (relative to what is detected in the absence of the candidate compound) is indicative of a modulation (e.g., stimulation or inhibition) of the interaction between the human caspase-14 protein and the target molecule. Modulation of the formation of complexes between the human caspase-14 protein and the target molecule can be quantitated using, for example, an immunoassay.

To perform cell free drug screening assays, it is desirable to immobilize either the human caspase-14 protein or its target molecule to facilitate separation of complexes from uncomplexed forms of one or both of the polypeptides, as well as to accommodate automation of the assay. Interaction (e.g., binding of) of human caspase-14 to a target molecule, in the presence and absence of a candidate compound, can be accomplished in any vessel suitable for containing the reactants. Examples of such vessels include microtitre plates, test tubes, and micro-centrifuge tubes. In one embodiment, a fusion polypeptide can be provided which adds a domain that allows the polypeptide to be bound to a matrix. For example, glutathione-S-transferase/caspase-14 fusion polypeptides can be adsorbed onto glutathione sepharose beads (Sigma Chemical, St. Louis, Mo.) or glutathione derivatized microtitre plates, which are then combined with the cell lysates (e.g., ³⁵S-labeled) and the candidate compound, and the mixture incubated under conditions conducive to complex formation (e.g., at physiological conditions for salt and pH). Following incubation, the beads are washed to remove any unbound label, and the matrix immobilized and radiolabel determined directly, or in the supernatant after the complexes are dissociated. Alternatively, the complexes can be dissociated from the matrix, separated by SDS-PAGE, and the level of human caspase-14-binding target found in the bead fraction quantitated from the gel using standard electrophoretic techniques.

Other techniques for immobilizing polypeptides on matrices can also be used in the drug screening assays of the invention. For example, either human caspase-14 or its target molecule can be immobilized utilizing conjugation of biotin and streptavidin. Biotinylated human caspase-14 molecules can be prepared from biotin-NHS (N-hydroxy-succinimide) using techniques well known in the art (e.g., biotinylation kit, Pierce Chemicals, Rockford, Ill.), and immobilized in the wells of streptavidin-coated 96 well plates (Pierce Chemical). Alternatively, antibodies reactive with human caspase-14 but which do not interfere with binding of the polypeptide to its target molecule can be derivatized to the wells of the plate, and human caspase-14 trapped in the wells by antibody conjugation. As described above, preparations of a human caspase-14-binding target and a candidate compound are incubated in the caspase-14-presenting wells of the plate, and the amount of complex trapped in the well can be quantitated. Methods for detecting such complexes, in addition to those described above for the GST-immobilized complexes, include immunodetection of complexes using antibodies reactive with the human caspase-14 target molecule, or which are reactive with human caspase-14 protein and compete with the target molecule; as well as enzyme-linked assays which rely on detecting an enzymatic activity associated with the target molecule.

In yet another embodiment, the invention provides a method for identifying a compound (e.g., a screening assay) capable of use in the treatment of a disorder characterized by (or associated with) aberrant or abnormal human caspase-14 nucleic acid expression or human caspase-14 protein activity. This method typically includes the step of assaying the ability of the compound or agent to modulate the expression of the human caspase-14 nucleic acid or the activity of the human caspase-14 protein thereby identifying a compound for treating a disorder characterized by aberrant or abnormal human caspase-14 nucleic acid expression or human caspase-14 protein activity. Disorders characterized by aberrant or abnormal human caspase-14 nucleic acid expression or human caspase-14 protein activity are described herein. Methods for assaying the ability of the compound or agent to modulate the expression of the human caspase-14 nucleic acid or activity of the human caspase-14 protein are typically cell-based assays. For example, cells which are sensitive to ligands which transduce signals via a pathway involving human caspase-14 can be induced to overexpress a human caspase-14 protein in the presence and absence of a candidate compound. Candidate compounds which produce a statistically significant change in human caspase-14-dependent responses (either stimulation or inhibition) can be identified. In one embodiment, expression of the human caspase-14 nucleic acid or activity of a human caspase-14 protein is modulated in cells and the effects of candidate compounds on the readout of interest (such as apoptosis) are measured. For example, the expression of genes which are up- or down-regulated in response to a human caspase-14-dependent signal cascade can be assayed. In preferred embodiments, the regulatory regions of such genes, e.g., the 5′ flanking promoter and enhancer regions, are operably linked to a detectable marker (such as luciferase) which encodes a gene product that can be readily detected. Phosphorylation of human caspase-14 or human caspase-14 target molecules can also be measured, for example, by immunoblotting.

Alternatively, modulators of human caspase-14 expression (e.g., compounds which can be used to treat a disorder characterized by aberrant or abnormal human caspase-14 nucleic acid expression or human caspase-14 protein activity) can be identified in a method wherein a cell is contacted with a candidate compound and the expression of human caspase-14 mRNA or polypeptide in the cell is determined. The level of expression of human caspase-14 mRNA or polypeptide in the presence of the candidate compound is compared to the level of expression of human caspase-14 mRNA or polypeptide in the absence of the candidate compound. The candidate compound can then be identified as a modulator of human caspase-14 nucleic acid expression based on this comparison and be used to treat a disorder characterized by aberrant human caspase-14 nucleic acid expression. For example, when expression of human caspase-14 mRNA or polypeptide is greater (statistically significantly greater) in the presence of the candidate compound than in its absence, the candidate compound is identified as a stimulator of human caspase-14 nucleic acid expression. Alternatively, when human caspase-14 nucleic acid expression is less (statistically significantly less) in the presence of the candidate compound than in its absence, the candidate compound is identified as an inhibitor of human caspase-14 nucleic acid expression. The level of human caspase-14 nucleic acid expression in the cells can be determined by methods described herein for detecting human caspase-14 mRNA or polypeptide.

In yet another aspect of the invention, the human caspase-14 proteins, or fragments thereof, can be used as “bait proteins” in a two-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO 94/10300), to identify other proteins, which bind to or interact with human caspase-14 (“human caspase-14-binding proteins” or “human caspase-14-bp”) and modulate human caspase-14 protein activity. Such human caspase-14-binding proteins are also likely to be involved in the propagation of signals by the human caspase-14 proteins as, for example, upstream or downstream elements of the human caspase-14 pathway.

The two-hybrid system is based on the modular nature of most transcription factors, which consist of separable DNA-binding and activation domains. Bartel, P. et al. “Using the Two-Hybrid System to Detect Protein-Protein Interactions” in Cellular Interactions in Development: A Practical Approach, Hartley, D. A. ed. (Oxford University Press, Oxford, 1993) pp. 153-179. Briefly, the assay utilizes two different DNA constructs. In one construct, the gene that codes for human caspase-14 is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a human caspase-14-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the protein which interacts with human caspase-14.

The invention further provides a method for detecting the presence of human caspase-14, or fragment thereof, in a biological sample. The method involves contacting the biological sample with a compound or an agent capable of detecting human caspase-14 protein or mRNA such that the presence of human caspase-14 is detected in the biological sample. A preferred agent for detecting human caspase-14 mRNA is a labeled or labelable nucleic acid probe capable of hybridizing to human caspase-14 mRNA. The nucleic acid probe can be, for example, the full-length human caspase-14 cDNA of SEQ ID NO:1, or a portion thereof, such as an oligonucleotide of at least 15, 30, 50, 100, 250 or 500 nucleotides in length and sufficient to specifically hybridize under stringent conditions to human caspase-14 mRNA. A preferred agent for detecting human caspase-14 protein is a labeled or labelable antibody capable of binding to human caspase-14 protein. Antibodies can be polyclonal, or more preferably, monoclonal. An intact antibody, or a fragment thereof (e.g., Fab or F(ab′)₂) can be used. The term “labeled or labelable”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The term “biological sample” is intended to include tissues, cells and biological fluids isolated from a subject, as well as tissues, cells and fluids present within a subject. That is, the detection method of the invention can be used to detect human caspase-14 mRNA or polypeptide in a biological sample in vitro as well as in vivo. For example, in vitro techniques for detection of human caspase-14 mRNA include Northern hybridizations and in situ hybridizations. In vitro techniques for detection of human caspase-14 protein include enzyme linked immunosorbent assays (ELISAs), Western blots, immunoprecipitations and immunofluorescence. Alternatively, human caspase-14 protein can be detected in vivo in a subject by introducing into the subject a labeled anti-human caspase-14 antibody. For example, the antibody can be labeled with a radioactive marker whose presence and location in a subject can be detected by standard imaging techniques.

The invention also encompasses kits for detecting the presence of human caspase-14 in a biological sample. For example, the kit can comprise a labeled or labelable compound or agent capable of detecting human caspase-14 protein or mRNA in a biological sample; means for determining the amount of human caspase-14 in the sample; and means for comparing the amount of human caspase-14 in the sample with a standard. The compound or agent can be packaged in a suitable container. The kit can further comprise instructions for using the kit to detect human caspase-14 mRNA or protein.

The methods of the invention can also be used to detect naturally occurring genetic mutations in a human caspase-14 gene, thereby determining if a subject with the mutated gene is at risk for a disorder characterized by aberrant or abnormal human caspase-14 nucleic acid expression or human caspase-14 protein activity as described herein. In preferred embodiments, the methods include detecting, in a sample of cells from the subject, the presence or absence of a genetic mutation characterized by at least one of an alteration affecting the integrity of a gene encoding a human caspase-14 protein, or the misexpression of the human caspase-14 gene. For example, such genetic mutations can be detected by ascertaining the existence of at least one of 1) a deletion of one or more nucleotides from a human caspase-14 gene; 2) an addition of one or more nucleotides to a human caspase-14 gene; 3) a substitution of one or more nucleotides of a human caspase-14 gene, 4) a chromosomal rearrangement of a human caspase-14 gene; 5) an alteration in the level of a messenger RNA transcript of a human caspase-14 gene, 6) aberrant modification of a human caspase-14 gene, such as of the methylation pattern of the genomic DNA, 7) the presence of a non-wild type splicing pattern of a messenger RNA transcript of a human caspase-14 gene, 8) a non-wild type level of a human caspase-14-polypeptide, 9) allelic loss of a human caspase-14 gene, and 10) inappropriate post-translational modification of a human caspase-14-polypeptide. As described herein, there are a large number of assay techniques known in the art which can be used for detecting mutations in a human caspase-14 gene.

In certain embodiments, detection of the mutation involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) Proc. Natl. Acad. Sci. USA 91:360-364), the latter of which can be particularly useful for detecting point mutations in the human caspase-14-gene (see Abravaya et al. (1995) Nucleic Acids Res. 23:675-682). This method can include the steps of collecting a sample of cells from a patient, isolating nucleic acid (e.g., genomic, mRNA or both) from the cells of the sample, contacting the nucleic acid sample with one or more primers which specifically hybridize to a human caspase-14 gene under conditions such that hybridization and amplification of the human caspase-14-gene (if present) occurs, and detecting the presence or absence of an amplification product, or detecting the size of the amplification product and comparing the length to a control sample.

In an alternative embodiment, mutations in a human caspase-14 gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, for example, U.S. Pat. No. 5,498,531) can be used to score for the presence of specific mutations by development or loss of a ribozyme cleavage site.

In yet another embodiment, any of a variety of sequencing reactions known in the art can be used to directly sequence the human caspase-14 gene and detect mutations by comparing the sequence of the sample human caspase-14 with the corresponding wild-type (control) sequence. Examples of sequencing reactions include those based on techniques developed by Maxam and Gilbert ((1977) Proc. Natl. Acad. Sci. USA 74:560) or Sanger ((1977) Proc. Natl. Acad. Sci. USA 74:5463). A variety of automated sequencing procedures can be utilized when performing the diagnostic assays ((1995) Biotechniques 19:448), including sequencing by mass spectrometry (see, e.g., PCT International Publication No. WO 94/16101; Cohen et al. (1996) Adv. Chromatogr. 36:127-162; and Griffin et al. (1993) Appl. Biochem. Biotechnol. 38:147-159).

Other methods for detecting mutations in the human caspase-14 gene include methods in which protection from cleavage agents is used to detect mismatched bases in RNA/RNA or RNA/DNA duplexes (Myers et al. (1985) Science 230:1242); Cotton et al. (1988) Proc. Natl. Acad. Sci. USA 85:4397; Saleeba et al. (1992) Methods Enzymol. 217:286-295), electrophoretic mobility of mutant and wild type nucleic acid is compared (Orita et al. (1989) Proc. Natl. Acad. Sci. USA 86:2766; Cotton (1993) Mutat. Res. 285:125-144; and Hayashi (1992) Genet. Anal. Tech. Appl. 9:73-79), and movement of mutant or wild-type fragments in polyacrylamide gels containing a gradient of denaturant is assayed using denaturing gradient gel electrophoresis (Myers et al. (1985) Nature 313:495). Examples of other techniques for detecting point mutations include, selective oligonucleotide hybridization, selective amplification, and selective primer extension.

The present invention is further illustrated by the following example which should not be construed as limiting in any way. The contents of all cited references, including literature references, issued patents, and published patent applications, as cited throughout this application are hereby expressly incorporated by reference.

### EXAMPLE

Isolation and Characterization of Human Caspase-14 cDNA

Attempts were made to clone the human caspase-14 cDNA (also known as MICE, for mini-ICE) using PCR primers based on the published predicted sequence disclosed in Van de Craen, M. et al. (1998) Cell Death Diff. 5(10):838-846. In this publication, the authors predicted the human caspase-14 sequence using the mouse sequence and a gene prediction software (GENSCAN) to analyze human genomic DNA. The primers that were used to attempt to PCR amplify human caspase-14 cDNA were based on these sequences and yet no PCR products of the full-length open reading frame (ORF) were obtained using a number of different cDNA libraries. These results suggested that the published predicted sequence for human caspase-14 was incorrect.

It was possible, however, to generate a shorter PCR product that contained the C-terminal portion of the ORF using the primers:

BBC-N 4634
5′ caspase-14 GGC CCT GCG AGC TAA GCC CAA GGT (SEQ ID NO:6)
BBC-N 4636
3′caspase-14 AAA AAG ATC TCT ACT GCA GAT ACA GCC GTT TCC GGA GGG
TGC TTT GGA T (SEQ ID NO:7)

The cDNA library used as the PCR template was a human fibroblast skin cDNA library (Clontech; catalog #HL10526), in which the mRNA source was cultured primary fibroblasts from a young male and the cDNAs were cloned into the EcoR1 cloning site of the Lambda gt11 cloning vector. This library was used for PCR along with the primer pair 4636/4634 (shown above) in a reaction mixture that that contained 1 μl of boiled library, 1 μl of 20 μM 4636 primer, 1 μl of 20 μM 4634 primer, 2.5 μl of 10 mM dNTPs, 10 μl of 10×PCR buffer containing MgCl₂, 1 μl of amplitaq enzyme and 83.5 μl of water. The amplification scheme was as follows: 94° C. for 30 seconds, 55° C. for 30 seconds, 72° C. for 2 minutes for 30 cycles, followed by 72° C. for 5 minutes for 1 cycle and then the reaction mixture was held at 4° C. This produced a 348 base pair fragment of human caspase-14 sequence. The identity of the PCR product was confirmed by DNA sequence analysis.

To obtain a full-length cDNA molecule, the 348 bp PCR product was labeled with ³²P using Amersham Multiprime labeling kit. This labeled probe was used for primary screening of the same fibroblast cDNA library with duplicate plaque lifts on NEN nylon 137 mm membrane circles. The filters were pre-wet with 2×SSC (as described in Maniatis, A Cloning Manual). Prehybridization was carried out for 2 hours at 42° C. in the following hybridization buffer: 6.66×SSPE (Maniatis, A Cloning Manual), 0.5% SDS, 50% formamide, 0.1 mg/ml salmon sperm DNA (pre-boiled and sheared). The first hybridization was carried out by addition of fresh hybridization buffer containing the boiled probe DNA and incubation continued at 42° C. overnight. The filters were first washed for 15 minutes at room temperature in 2×SSC, 0.1% SDS. The second wash was for 3 hours at 65° C. in IX SSC, 0.1% SDS. The final wash was for 1 hour at 65° C. in 1×SSC, 0.05% SDS. The filters were then exposed to Kodak XAR5 film for autoradiography. Phage DNA was prepared from 21 first round positive hybridizing plaques. These were screened for caspase-14 sequences using the same PCR primers that generated the 346 bp fragment corresponding to the caspase-143′ end. One phage DNA sample was identified to contain template for the 348 bp 3′ end fragment by PCR. The positive plaques were taken into a second round of hybridization screening using the same probe. Probe DNA was prepared as previous. The second round hybridization was carried out using the same conditions as the primary screen. Two plates had enriched plaques and phage DNA was prepared using 2 plaques from each plate. By caspase-14 PCR, only 2 plaques from one plate had the 348 bp insert. Phage DNA was prepared and their DNA sequences were shown to be full-length human caspase-14.

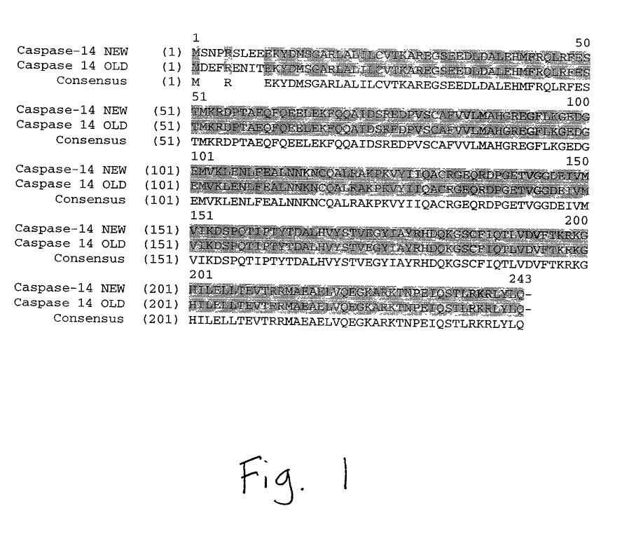

The determined DNA sequence for the full-length human caspase-14 cDNA is shown in SEQ ID NO:1. This human caspase-14 cDNA comprises a 5′ untranslated region corresponding to nucleotide positions 1-192, a coding region corresponding to nucleotide positions 193-918 and a 3′ untranslated region corresponding to nucleotide positions 919-1003. The predicted amino acid sequence encoded by the cDNA is shown in SEQ ID NO:2 and comprises a 242 amino acid protein. This amino acid sequence does not match that of the published predicted human caspase-14 sequence disclosed in Van de Craen, M. et al. (1998) Cell Death Dif. 5:838-846. More specifically, the amino terminal portions of the sequences are different. A comparison of the sequence of SEQ ID NO:2 (referred to as “Caspase-14 NEW”) with that of the published human caspase-14 sequence (referred to as “Caspase-14 OLD”; SEQ ID NO:9) is shown in FIG. 1, along with a consensus sequence. The human caspase-14 of SEQ ID NO:2 has an amino terminal sequence of Met-Ser-Asn-Pro-Arg-Ser-Leu-Glu-Glu (SEQ ID NO:4), whereas the published human caspase-14 has an amino terminal sequence of Met-Asp-Glu-Phe-Arg-Glu-Asn-Ile-Thr (SEQ ID NO:5). This explains the lack of full-length PCR product using PCR primers based on the published sequence.

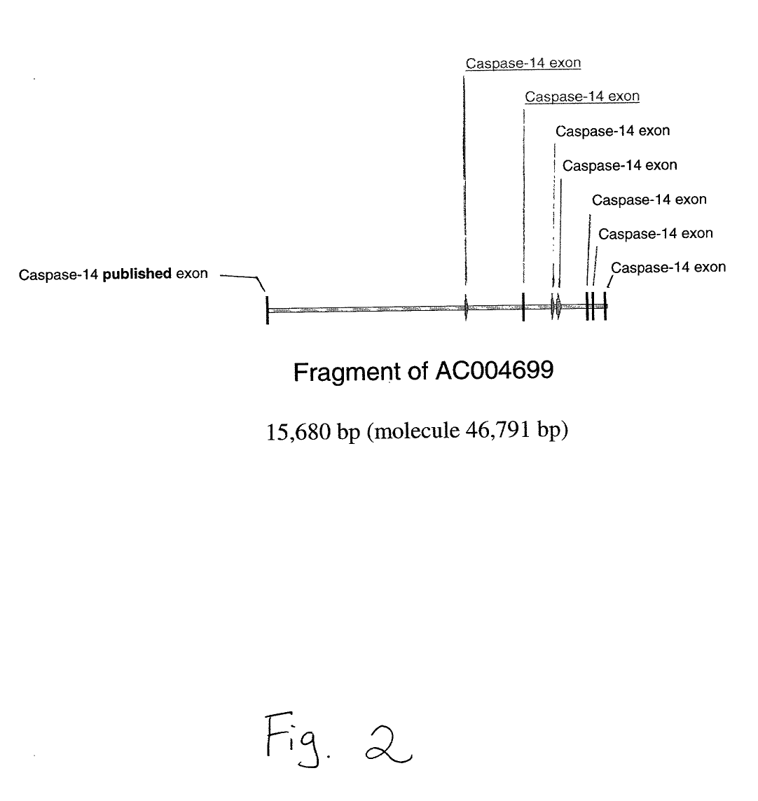

The differences at the amino termini are due to the choice of exons for the corresponding amino acid sequence. This is even more clear when the exon structure of the genomic sequence is examined, as illustrated in FIG. 2. The predicted exon (according to Van de Craen, M. et al. (1998) Cell Death Dif. 5:838-846) that contains the amino terminal sequence of the predicted published sequence is located over 10 kilobases away from the next exon. The cDNA sequence derived from the clone described herein has two exons that are much closer to the remainder of the gene. The first one contains untranslated sequence and the second contains the start codon and the first 8 amino acids. The last five exons are the same as the published sequence.

Equivalents

Those skilled in the art will recognize, or be able to ascertain using no more than routine experimentation, many equivalents to the specific embodiments of the invention described herein. Such equivalents are intended to be encompassed by the following claims.

We claim:

1. An isolated human caspase-14 nucleic acid, wherein the nucleic acid comprises a coding region encoding human caspase-14 and the coding region comprises a nucleotide sequence ATG AGC AAT CCG CGG TCT TTG GAA GAG (SEQ ID NO:3) at its 5′ end, or a complement thereof.

2. The isolated nucleic acid of claim 1, wherein the nucleic acid comprises the coding region of the nucleotide sequence of SEQ ID NO:1 (nucleotide positions 193-918), or a complement thereof.

3. The isolated nucleic acid of claim 1, wherein the nucleic acid comprises the nucleotide sequence of SEQ ID NO:1, or a complement thereof.

4. The isolated nucleic acid of claim 1, wherein the nucleic acid has at least 95% nucleotide identity with the nucleotide sequence of SEQ ID NO:1, or a complement thereof.

5. The isolated nucleic acid of claim 1, wherein the nucleic acid has at least 97% nucleotide identity with the nucleotide sequence of SEQ ID NO:1, or a complement thereof.

6. The isolated nucleic acid of claim 1, wherein the nucleic acid has at least 99% nucleotide identity with the nucleotide sequence of SEQ ID NO:1, or a complement thereof.

7. An isolated human caspase-14 nucleic acid, wherein the nucleic acid comprises a coding region encoding human caspase-14 and the coding region encodes an amino acid sequence Met Ser Asn Pro Arg Ser Leu Glu Glu (SEQ ID NO:4) at its 5′ end, or a complement thereof.

8. The isolated nucleic acid of claim 7, wherein the nucleic acid encodes the amino acid sequence of SEQ ID NO:2, or a complement thereof.

9. The isolated nucleic acid of claim 7, wherein the nucleic acid encodes an amino acid sequence having at least 95% amino acid identity with the amino acid sequence of SEQ ID NO:2, or a complement thereof.

10. The isolated nucleic acid of claim 7, wherein the nucleic acid encodes an amino acid sequence having at least 97% amino acid identity with the amino acid sequence of SEQ ID NO:2, or a complement thereof.

11. The isolated nucleic acid of claim 7, wherein the nucleic acid encodes an amino acid sequence having at least 99% amino acid identity with the amino acid sequence of SEQ ID NO:2, or a complement thereof.

12. The isolated nucleic acid of claim 1, which comprises a cDNA sequence.

13. An isolated antisense nucleic acid comprising at least a portion of a complement of the nucleotide sequence ATG AGC AAT CCG CGG TCT TTG GAA GAG (SEQ ID NO:3).

14. A kit comprising a compound which selectively hybridizes to a nucleic acid of claim 1 and instructions for use.

15. An expression vector comprising the nucleic acid of claim 1.

16. A host cell comprising the expression vector of claim 15.

17. A method for producing human caspase-14 protein comprising culturing the host cell of claim 16 in a suitable culture medium until human caspase-14 protein is produced.

18. The method of claim 17, further comprising isolating the human caspase-14 protein from the cells or the culture medium.

19. An isolated human caspase-14 protein comprising an amino acid sequence Met Ser Asn Pro Arg Ser Leu Glu Glu (SEQ ID NO:4) at its amino terminus.

20. The isolated protein of claim 19, which comprises the amino acid sequence of SEQ ID NO:2.

21. The isolated protein of claim 19, which comprises an amino acid sequence having at least 95% amino acid identity with the amino acid sequence of SEQ ID NO:2.

22. The isolated protein of claim 19, which comprises an amino acid sequence having at least 97% amino acid identity with the amino acid sequence of SEQ ID NO:2.

23. The isolated protein of claim 19, which comprises an amino acid sequence having at least 99% amino acid identity with the amino acid sequence of SEQ ID NO:2.

24. The isolated protein of claim 19, which comprises an amino acid sequence Met Ser Asn Pro Arg Ser Leu Glu Glu (SEQ ID NO:4) at its amino terminus and further has at least 95% amino acid identity with amino acid positions 1-154 of SEQ ID NO:2.

25. The isolated protein of claim 19, which is a fusion protein comprising the human caspase-14 protein operatively linked to a non-caspase-14 protein or polypeptide.

26. A kit comprising a compound which selectively binds to the human caspase-14 protein of claim 19 and instructions for use.

27. A pharmaceutical composition comprising a pharmaceutically acceptable carrier and the human caspase-14 protein of claim 19.

28. An antibody that binds to the human caspase-14 protein of claim 19, wherein the antibody binds to the amino acid sequence Met Ser Asn Pro Arg Ser Leu Glu Glu (SEQ ID NO:4).

29. The antibody of claim 28, which is a monoclonal human antibody.

30. The antibody of claim 28, which is linked to a therapeutic agent.

31. The antibody of claim 30, wherein the therapeutic agent is a cytotoxic agent.

32. The antibody of claim 30, wherein the therapeutic agent is a radioactive material.

33. A pharmaceutical composition comprising a pharmaceutically acceptable carrier and the antibody of claim 28.

34. The pharmaceutical composition of claim 33, wherein the antibody is a monoclonal human antibody.

35. A non-human transgenic animal having cells comprising the nucleic acid of claim 1.

36. A method for identifying a compound which is a modulator of human caspase-14 activity comprising:
a) contacting the human caspase-14 protein with a caspase-14 substrate under conditions suitable for proteolysis; and
b) determining the ability of the human caspase-14 protein to cleave the caspase-14 substrate,
thereby identifying a compound which is a modulator of human caspase-14 activity.

37. The method of claim 36, wherein the compound is an inhibitor of caspase-14 activity.

38. The method of claim 36, wherein the compound is an activator of caspase-14 activity.

39. A method for identifying a compound which binds the human caspase-14 protein, the method comprising:
a) contacting the human caspase-14 protein, or a cell expressing the human caspase-14 protein, with a test compound under conditions suitable for binding; and
b) detecting binding of the test compound to the human caspase-14 protein.

40. A method for identifying a compound which modulates the interaction of the human caspase-14 protein with a target molecule comprising:
a) contacting, in the presence of the compound, the human caspase-14 protein and the target molecule under conditions which allow binding of the target molecule to the human caspase-14 protein to form a complex; and
b) detecting the formation of a complex of the human caspase-14 protein and the target molecule, in which the ability of the compound to modulate interaction between the human caspase-14 protein and the target molecule is indicated by a change in complex formation as compared to the amount of complex formed in the absence of the compound.

41. A method for identifying a compound capable of treating a disorder characterized by aberrant or abnormal human caspase-14 nucleic acid expression or human caspase-14 activity comprising:
a) contacting a cell which expresses the human caspase-14 protein with a test compound; and
b) assaying the ability of the test compound to modulate the expression of human caspase-14 nucleic acid or the activity of a human caspase-14 protein, thereby identifying a compound capable of treating a disorder characterized by aberrant or abnormal human caspase-14 nucleic acid expression or human caspase-14 activity.

42. A method for modulating apoptosis in a cell comprising contacting a cell with a caspase-14 modulator, thereby modulating apoptosis in the cell.
